(19) United States
(12) Patent Application Publication
(10) Pub. No.: US 2012/0093734 A1
(43) Pub. Date: Apr. 19, 2012

(54) PROTEIN-BASED METHODS AND COMPOSITIONS FOR THE DIAGNOSIS OF COLORECTAL ADENOCARCINOMA

(75) Inventors: Meike De Wit, Utrecht (NL); Remondus Johannes Adriaan Fijneman, Westzaan (NL); Cornelia Ramona Jimenez, Amsterdam (NL); Gerrit Albert Meijer, Hattem (NL)

(73) Assignee: VERENIGING VOOR CHRISTELIJK HOGER ONDERWIJS, WETTENSCHAPPELIJK ONDERZOEK EN PATIENTENZORG, AMSTERDAM (NL)

(21) Appl. No.: 13/262,711

(22) PCT Filed: Mar. 31, 2010
(86) PCT No.: PCT/IB2010/051395

§ 371 Date: Oct. 3, 2011

(30) Foreign Application Priority Data
Apr. 7, 2009 (EP) 09157464.0

(51) Int. Cl. G01N 33/82; C07K 16/18; A61K 49/16; C07K 14/47

(52) U.S. Cl. 424/9.341; 530/350; 530/387.9; 435/7.23

(57) ABSTRACT

Protein-based methods and compositions for the diagnosis of colorectal adenocarcinoma are disclosed. A method for identifying cell-surface proteins, which are transmembrane proteins or proteins with a signal peptide and which are over-expressed in colorectal cancer (CRC) is disclosed. Biomarkers found with this method, diagnostic methods using them and contrast agents directed to them for use in magnetic resonance imaging (MRI) and/or magentic photon imaging (MPI) are disclosed. The methods and biomarkers allow for differentiating progressive (high-risk) CRC (adenocarcinomas) from non-progressive (low-risk) colorectal adenomas.

### SUBJECT OF THE INVENTION

The present invention relates to contrast agents, diagnostic markers and methods for detecting colorectal adenocarcinoma.

### BACKGROUND OF THE INVENTION

Most cancers are epithelial in origin and arise through stepwise progression from normal cells, through dysplasia, into malignant cells that invade surrounding tissues and have metastatic potential. Colorectal cancer (CRC; also referred to as colon cancer or large bowl cancer) is one prominent type of cancer undergoing such tumour progression.

CRC includes cancerous growth in the colon, rectum and appendix. It is one of the most significant human cancers with an incidence of about 1,000,000 new cases worldwide every year. Thus, CRC is the third most common cancer and the fourth leading cause of cancer-related deaths in the world (the second leading cause in the Western world; reviewed, e.g. in Gryfe, R. et al. (1997) Curr. Probl. Cancer 21, 233-300; Petersen, G. M. et al (1999) Cancer 86, 2540-2550.

CRC is curable if diagnosed at an early stage (i.e. stage I) of tumour development. The five-year survival rate for early stage CRC is >90%. At this early stage, most patients have no phenotypic symptoms of the disease. Early detection can markedly improve chances of long-term survival, as the five-year survival rate for CRC at a late stage (i.e. stage IV) of tumour development is only <5%. More than 95% of the cases of CRC are manifested as adenocarcinomas (Muto, T. et al. (1975) Cancer 36, 2251-2270; Fearon, E. R. et al. (1990) Cell 61, 759 767).

In the past, screenings have been undertaken to identify genes which are implicated in the progression from low-risk colorectal adenomas to high-risk adenocarcinomas. To this end, genes were identified, the expression of which is either up- or down-regulated in colorectal adenocarcinomas versus adenomas (see e.g. Carvalho et al. (2009) Gut 58, 79-89).

Currently, colonoscopy is the standard screening modality for CRC having the highest sensitivity and specificity to detect colorectal tumours among techniques that are currently applied.

Nevertheless, screening by colonoscopy may have disadvantages. Among those are (i) that colonoscopy is an invasive technique being a limiting factor when CRC screening is offered for asymptomatic individuals, (ii) that there is a small but significant risk of bowel perforation as a consequence of colonoscopy and (iii) that colonoscopy cannot discriminate between non-progressive (low-risk) and progressive (high-risk) colon tumour lesions.

Thus, there is a continuing need for agents, compositions, markers and methods that allow diagnosis of CRC.

### OBJECTIVE AND SUMMARY OF THE INVENTION

It is one objective of the present invention to provide for methods that allow diagnosing CRC. It is a further objective of the present invention to provide for markers that can be used for diagnosing CRC.

Yet another objective of the present invention is concerned with the provision of contrast agents which can be used in the detection of CRC.

These and other objectives as they will become apparent from the ensuing description hereinafter form the subject matter of the independent claims. Some of the preferred embodiments of the present invention form the subject matter of the dependent claims.

The present invention in one embodiment thus relates to a diagnostic marker, preferably for detecting CRC comprising at least one polypeptide of the group consisting of:
A polypeptide of SEQ ID No.: 1;
A polypeptide of SEQ ID No.: 2;
A polypeptide of SEQ ID No.: 3;
A polypeptide of SEQ ID No.: 4;
A polypeptide of SEQ ID No.: 5;
A polypeptide of SEQ ID No.: 6;
A polypeptide of SEQ ID No.: 7;
A polypeptide of SEQ ID No.: 8;
A polypeptide of SEQ ID No.: 9;
A polypeptide of SEQ ID No.: 10;
A polypeptide of SEQ ID No.: 11;
A polypeptide of SEQ ID No.: 12;
A polypeptide of SEQ ID No.: 13;
A polypeptide of SEQ ID No.: 14;
A polypeptide of SEQ ID No.: 15;
A polypeptide of SEQ ID No.: 16;
A polypeptide of SEQ ID No.: 17;
A polypeptide of SEQ ID No.: 18;
A polypeptide of SEQ ID No.: 19;
A polypeptide of SEQ ID No.: 20;
A polypeptide of SEQ ID No.: 21;
A polypeptide of SEQ ID No.: 22;
A polypeptide of SEQ ID No.: 23;
A polypeptide of SEQ ID No.: 24;
A polypeptide of SEQ ID No.: 25;
A polypeptide of SEQ ID No.: 26;
A polypeptide of SEQ ID No.: 27;
A polypeptide of SEQ ID No.: 28;
A polypeptide of SEQ ID No.: 29;
A polypeptide of SEQ ID No.: 30;
A polypeptide of SEQ ID No.: 31; and/or
A polypeptide of SEQ ID No.: 32.

The present invention in the further embodiments relates to the use of at least one polypeptide selected from the group consisting of:
A polypeptide of SEQ ID No.: 1;
A polypeptide of SEQ ID No.: 2;
A polypeptide of SEQ ID No.: 3;
A polypeptide of SEQ ID No.: 4;
A polypeptide of SEQ ID No.: 5;
A polypeptide of SEQ ID No.: 6;
A polypeptide of SEQ ID No.: 7;
A polypeptide of SEQ ID No.: 8;
A polypeptide of SEQ ID No.: 9;
A polypeptide of SEQ ID No.: 10;
A polypeptide of SEQ ID No.: 11;
A polypeptide of SEQ ID No.: 12;
A polypeptide of SEQ ID No.: 13;
A polypeptide of SEQ ID No.: 14;
A polypeptide of SEQ ID No.: 15;
A polypeptide of SEQ ID No.: 16;
A polypeptide of SEQ ID No.: 17;
A polypeptide of SEQ ID No.: 18;
A polypeptide of SEQ ID No.: 19;
A polypeptide of SEQ ID No.: 20;
A polypeptide of SEQ ID No.: 21;
A polypeptide of SEQ ID No.: 22;
A polypeptide of SEQ ID No.: 23;
A polypeptide of SEQ ID No.: 24;
A polypeptide of SEQ ID No.: 25;
A polypeptide of SEQ ID No.: 26;
A polypeptide of SEQ ID No.: 27;
A polypeptide of SEQ ID No.: 28;
A polypeptide of SEQ ID No.: 29;
A polypeptide of SEQ ID No.: 30;
A polypeptide of SEQ ID No.: 31, and/or
A polypeptide of SEQ ID No.: 32.
   as a diagnostic marker, preferably for detecting CRC.

In one embodiment, such use is performed outside the human or animal body.

Yet another aspect of the present invention relates to a contrast agent, optionally for use in magnetic resonance imaging (MRI) and/or magnetic photon imaging (MPI) comprising at least at least one compound being capable of interacting with a polypeptide selected from the group consisting of:
A polypeptide of SEQ ID No.: 1;
A polypeptide of SEQ ID No.: 2;
A polypeptide of SEQ ID No.: 3;
A polypeptide of SEQ ID No.: 4;
A polypeptide of SEQ ID No.: 5;
A polypeptide of SEQ ID No.: 6;
A polypeptide of SEQ ID No.: 7;
A polypeptide of SEQ ID No.: 8;
A polypeptide of SEQ ID No.: 9;
A polypeptide of SEQ ID No.: 10;
A polypeptide of SEQ ID No.: 11;
A polypeptide of SEQ ID No.: 12;
A polypeptide of SEQ ID No.: 13;
A polypeptide of SEQ ID No.: 14;
A polypeptide of SEQ ID No.: 15;
A polypeptide of SEQ ID No.: 16;
A polypeptide of SEQ ID No.: 17;
A polypeptide of SEQ ID No.: 18;
A polypeptide of SEQ ID No.: 19;
A polypeptide of SEQ ID No.: 20;
A polypeptide of SEQ ID No.: 21;
A polypeptide of SEQ ID No.: 22;
A polypeptide of SEQ ID No.: 23;
A polypeptide of SEQ ID No.: 24;
A polypeptide of SEQ ID No.: 25;
A polypeptide of SEQ ID No.: 26;
A polypeptide of SEQ ID No.: 27;
A polypeptide of SEQ ID No.: 28;
A polypeptide of SEQ ID No.: 29;
A polypeptide of SEQ ID No.: 30;
A polypeptide of SEQ ID No.: 31; and/or A polypeptide of SEQ ID No.: 32.

In a preferred embodiment such compounds are coupled to marker molecules which preferably are detectable by MRI or MPI.

The compounds may preferably be antibodies.

Such contrast agents may preferably be used for detecting CRC.

In a further embodiment the present invention relates to use of at least one antibody capable of interacting with a polypeptide selected from the group consisting of:
A polypeptide of SEQ ID No.: 1;
A polypeptide of SEQ ID No.: 2;
A polypeptide of SEQ ID No.: 3;
A polypeptide of SEQ ID No.: 4;
A polypeptide of SEQ ID No.: 5;
A polypeptide of SEQ ID No.: 6;
A polypeptide of SEQ ID No.: 7;
A polypeptide of SEQ ID No.: 8;
A polypeptide of SEQ ID No.: 9;
A polypeptide of SEQ ID No.: 10;
A polypeptide of SEQ ID No.: 11;
A polypeptide of SEQ ID No.: 12;
A polypeptide of SEQ ID No.: 13;
A polypeptide of SEQ ID No.: 14;
A polypeptide of SEQ ID No.: 15;
A polypeptide of SEQ ID No.: 16;
A polypeptide of SEQ ID No.: 17;
A polypeptide of SEQ ID No.: 18;
A polypeptide of SEQ ID No.: 19;
A polypeptide of SEQ ID No.: 20;
A polypeptide of SEQ ID No.: 21;
A polypeptide of SEQ ID No.: 22;
A polypeptide of SEQ ID No.: 23;
A polypeptide of SEQ ID No.: 24;
A polypeptide of SEQ ID No.: 25;
A polypeptide of SEQ ID No.: 26;
A polypeptide of SEQ ID No.: 27;
A polypeptide of SEQ ID No.: 28;
A polypeptide of SEQ ID No.: 29;
A polypeptide of SEQ ID No.: 30;
A polypeptide of SEQ ID No.: 31; and/or
A polypeptide of SEQ ID No.: 32
   as a contrast agent, optionally suitable for MRI and/or MPI.

Such contrast agents may preferably be used for detecting CRC.

Another embodiment of the present invention relates to a method of diagnosing CRC comprising at least the following steps:

a) Obtaining at least one sample from at least one human or animal individual suspected to suffer from ongoing or imminent CRC development;

b) Testing in said at least one sample for expression of at least one polypeptide selected from the group consisting of:
A polypeptide of SEQ ID No.: 1;
A polypeptide of SEQ ID No.: 2;
A polypeptide of SEQ ID No.: 3;
A polypeptide of SEQ ID No.: 4;
A polypeptide of SEQ ID No.: 5;
A polypeptide of SEQ ID No.: 6;
A polypeptide of SEQ ID No.: 7;
A polypeptide of SEQ ID No.: 8;
A polypeptide of SEQ ID No.: 9;
A polypeptide of SEQ ID No.: 10;
A polypeptide of SEQ ID No.: 11;
A polypeptide of SEQ ID No.: 12;
A polypeptide of SEQ ID No.: 13;
A polypeptide of SEQ ID No.: 14;
A polypeptide of SEQ ID No.: 15;
A polypeptide of SEQ ID No.: 16;
A polypeptide of SEQ ID No.: 17;
A polypeptide of SEQ ID No.: 18;
A polypeptide of SEQ ID No.: 19;
A polypeptide of SEQ ID No.: 20;
A polypeptide of SEQ ID No.: 21;
A polypeptide of SEQ ID No.: 22;
A polypeptide of SEQ ID No.: 23;
A polypeptide of SEQ ID No.: 24;
A polypeptide of SEQ ID No.: 25;
A polypeptide of SEQ ID No.: 26;
A polypeptide of SEQ ID No.: 27;
A polypeptide of SEQ ID No.: 28;
A polypeptide of SEQ ID No.: 29;
A polypeptide of SEQ ID No.: 30;
A polypeptide of SEQ ID No.: 31; and/or
A polypeptide of SEQ ID No.: 32;

c) Testing in at least one control sample obtained from at least one human or animal individual not suffering from ongoing or imminent CRC development for expression of at least one polypeptide selected from the group consisting of:
A polypeptide of SEQ ID No.: 1;
A polypeptide of SEQ ID No.: 2;
A polypeptide of SEQ ID No.: 3;
A polypeptide of SEQ ID No.: 4;
A polypeptide of SEQ ID No.: 5;
A polypeptide of SEQ ID No.: 6;
A polypeptide of SEQ ID No.: 7;
A polypeptide of SEQ ID No.: 8;
A polypeptide of SEQ ID No.: 9;
A polypeptide of SEQ ID No.: 10;
A polypeptide of SEQ ID No.: 11;
A polypeptide of SEQ ID No.: 12;
A polypeptide of SEQ ID No.: 13;
A polypeptide of SEQ ID No.: 14;
A polypeptide of SEQ ID No.: 15;
A polypeptide of SEQ ID No.: 16;
A polypeptide of SEQ ID No.: 17;
A polypeptide of SEQ ID No.: 18;
A polypeptide of SEQ ID No.: 19;
A polypeptide of SEQ ID No.: 20;
A polypeptide of SEQ ID No.: 21;
A polypeptide of SEQ ID No.: 22;
A polypeptide of SEQ ID No.: 23;
A polypeptide of SEQ ID No.: 24;
A polypeptide of SEQ ID No.: 25;
A polypeptide of SEQ ID No.: 26;
A polypeptide of SEQ ID No.: 27;
A polypeptide of SEQ ID No.: 28;
A polypeptide of SEQ ID No.: 29;
A polypeptide of SEQ ID No.: 30;
A polypeptide of SEQ ID No.: 31; and/or
A polypeptide of SEQ ID No.: 32;

d) Determining difference in expression of steps b) and d);

e) Deciding on the presence or imminence of colorectal cancer development based on the results obtained in step d).

In one embodiment, steps b), c), d) and/or e) of this method of diagnosis are performed outside the human or animal body.

Yet another aspect of the present invention relates to a method of diagnosing colorectal cancer comprising at least the following steps:

a) Testing in at least one human or animal individual suspected to suffer from ongoing or imminent CRC development for expression of at least one polypeptide selected from the group consisting of:
A polypeptide of SEQ ID No.: 1;
A polypeptide of SEQ ID No.: 2;
A polypeptide of SEQ ID No.: 3;
A polypeptide of SEQ ID No.: 4;
A polypeptide of SEQ ID No.: 5;
A polypeptide of SEQ ID No.: 6;
A polypeptide of SEQ ID No.: 7;
A polypeptide of SEQ ID No.: 8;
A polypeptide of SEQ ID No.: 9;
A polypeptide of SEQ ID No.: 10;
A polypeptide of SEQ ID No.: 11;
A polypeptide of SEQ ID No.: 12;
A polypeptide of SEQ ID No.: 13;
A polypeptide of SEQ ID No.: 14;
A polypeptide of SEQ ID No.: 15;
A polypeptide of SEQ ID No.: 16;
A polypeptide of SEQ ID No.: 17;
A polypeptide of SEQ ID No.: 18;
A polypeptide of SEQ ID No.: 19;
A polypeptide of SEQ ID No.: 20;
A polypeptide of SEQ ID No.: 21;
A polypeptide of SEQ ID No.: 22;
A polypeptide of SEQ ID No.: 23;
A polypeptide of SEQ ID No.: 24;
A polypeptide of SEQ ID No.: 25;
A polypeptide of SEQ ID No.: 26;
A polypeptide of SEQ ID No.: 27;
A polypeptide of SEQ ID No.: 28;
A polypeptide of SEQ ID No.: 29;
A polypeptide of SEQ ID No.: 30;
A polypeptide of SEQ ID No.: 31; and/or
A polypeptide of SEQ ID No.: 32;

b) Comparing expression as determined in step a) with expression of at least one polypeptide selected from the group consisting of:
A polypeptide of SEQ ID No.: 1;
A polypeptide of SEQ ID No.: 2;
A polypeptide of SEQ ID No.: 3;
A polypeptide of SEQ ID No.: 4;
A polypeptide of SEQ ID No.: 5;
A polypeptide of SEQ ID No.: 6;
A polypeptide of SEQ ID No.: 7;
A polypeptide of SEQ ID No.: 8;
A polypeptide of SEQ ID No.: 9;
A polypeptide of SEQ ID No.: 10;
A polypeptide of SEQ ID No.: 11;
A polypeptide of SEQ ID No.: 12;
A polypeptide of SEQ ID No.: 13;
A polypeptide of SEQ ID No.: 14;
A polypeptide of SEQ ID No.: 15;
A polypeptide of SEQ ID No.: 16;
A polypeptide of SEQ ID No.: 17;
A polypeptide of SEQ ID No.: 18;
A polypeptide of SEQ ID No.: 19;
A polypeptide of SEQ ID No.: 20;
A polypeptide of SEQ ID No.: 21;
A polypeptide of SEQ ID No.: 22;
A polypeptide of SEQ ID No.: 23;
A polypeptide of SEQ ID No.: 24;
A polypeptide of SEQ ID No.: 25;
A polypeptide of SEQ ID No.: 26;
A polypeptide of SEQ ID No.: 27;
A polypeptide of SEQ ID No.: 28;
A polypeptide of SEQ ID No.: 29;
A polypeptide of SEQ ID No.: 30;
A polypeptide of SEQ ID No.: 31; and/or
A polypeptide of SEQ ID No.: 32;
   as determined for human or animal individuals not suffering from ongoing or imminent CRC development

c) Deciding on the presence or imminence of CRC development based on the results obtained in step b).

Another aspect of the present invention relates to a method of data acquisition comprising at least the following steps:

a) Testing in at least one human or animal individual suspected to suffer from ongoing or imminent CRC development for expression of at least one polypeptide selected from the group consisting of:
A polypeptide of SEQ ID No.: 1;
A polypeptide of SEQ ID No.: 2;
A polypeptide of SEQ ID No.: 3;
A polypeptide of SEQ ID No.: 4;
A polypeptide of SEQ ID No.: 5;
A polypeptide of SEQ ID No.: 6;
A polypeptide of SEQ ID No.: 7;
A polypeptide of SEQ ID No.: 8;
A polypeptide of SEQ ID No.: 9;
A polypeptide of SEQ ID No.: 10;
A polypeptide of SEQ ID No.: 11;
A polypeptide of SEQ ID No.: 12;
A polypeptide of SEQ ID No.: 13;
A polypeptide of SEQ ID No.: 14;
A polypeptide of SEQ ID No.: 15;
A polypeptide of SEQ ID No.: 16;
A polypeptide of SEQ ID No.: 17;
A polypeptide of SEQ ID No.: 18;
A polypeptide of SEQ ID No.: 19;
A polypeptide of SEQ ID No.: 20;
A polypeptide of SEQ ID No.: 21;
A polypeptide of SEQ ID No.: 22;
A polypeptide of SEQ ID No.: 23;
A polypeptide of SEQ ID No.: 24;
A polypeptide of SEQ ID No.: 25;
A polypeptide of SEQ ID No.: 26;
A polypeptide of SEQ ID No.: 27;
A polypeptide of SEQ ID No.: 28;
A polypeptide of SEQ ID No.: 29;
A polypeptide of SEQ ID No.: 30;
A polypeptide of SEQ ID No.: 31; and/or
A polypeptide of SEQ ID No.: 32;

b) Comparing expression as determined in step a) with expression of at least one polypeptide selected from the group consisting of:
A polypeptide of SEQ ID No.: 1;
A polypeptide of SEQ ID No.: 2;
A polypeptide of SEQ ID No.: 3;
A polypeptide of SEQ ID No.: 4;
A polypeptide of SEQ ID No.: 5;
A polypeptide of SEQ ID No.: 6;
A polypeptide of SEQ ID No.: 7;
A polypeptide of SEQ ID No.: 8;
A polypeptide of SEQ ID No.: 9;
A polypeptide of SEQ ID No.: 10;
A polypeptide of SEQ ID No.: 11;
A polypeptide of SEQ ID No.: 12;
A polypeptide of SEQ ID No.: 13;
A polypeptide of SEQ ID No.: 14;
A polypeptide of SEQ ID No.: 15;
A polypeptide of SEQ ID No.: 16;
A polypeptide of SEQ ID No.: 17;
A polypeptide of SEQ ID No.: 18;
A polypeptide of SEQ ID No.: 19;
A polypeptide of SEQ ID No.: 20;
A polypeptide of SEQ ID No.: 21;
A polypeptide of SEQ ID No.: 22;
A polypeptide of SEQ ID No.: 23;
A polypeptide of SEQ ID No.: 24;
A polypeptide of SEQ ID No.: 25;
A polypeptide of SEQ ID No.: 26;
A polypeptide of SEQ ID No.: 27;
A polypeptide of SEQ ID No.: 28;
A polypeptide of SEQ ID No.: 29;
A polypeptide of SEQ ID No.: 30;
A polypeptide of SEQ ID No.: 31; and/or
A polypeptide of SEQ ID No.: 32;
   as determined for human or animal individuals not suffering from ongoing or imminent CRC development.

Further, an embodiment of the present invention relates to a method of identifying at least one target molecule suitable as diagnostic marker for colorectal cancer, wherein the method comprises at least the following steps:

a) Obtaining cells from different human individuals all suffering from colorectal cancer and/or obtaining colorectal cancer cell lines;

b) Labeling polypeptides on the surface of said cells;

c) Isolating labelled polypeptides from non-labelled polypeptides;

d) Identification of isolated labelled polypeptides from the cell surface polypeptide-fraction of step c),

e) Of the polypeptides of step d), selecting those isolated labelled polypeptides for which genomic expression data indicate an increased expression compared to cells obtained from healthy individuals;

f) Of the polypeptides selected in step e), further selecting those isolated labelled polypeptides which comprise at least one transmembrane domain and/or at least one signal peptide;

g) Of the polypeptides selected in step f), further selecting those polypeptides with a positive RSC;

h) Of the polypeptides selected in step g), further selecting those polypeptides found within at least 70% of tested cells from different human individuals;

i) Of the polypeptides selected in step h), further selecting those polypeptides for which histological analysis confirms localization to the plasma membrane.

The aforementioned diagnostic markers, contrast agents, uses and methods are suitable for differentiating progressive (high-risk) CRC (adenocarcinomas) from non-progressive (low-risk) colorectal adenomas.

Further, the diagnostic markers, contrast agents, uses and methods of the present invention may be particularly suitable for non-invasive molecular imaging, for instance by MRI, for diagnosis of CRC.

Other embodiments of the present invention will become apparent from the detailed description hereinafter.

### FIGURE LEGENDS

FIG. 1 depicts SDS-PAGE analysis of Neuravidine-purified biotin-labelled cell surface or intracellular fractions of colorectal cancer cell lines Colo 205, HT-29, Caco2, RKO and HCT116. The two lanes of intracellular fractions correspond to the fractions obtained from the CRC cell lines Colo 205, HT29 and Caco2 (left lane) and RKO and HCT116 (right lane).

FIG. 2 depicts histological analysis of PRNP (SEQ ID NO: 6).

FIG. 3 depicts histological detection of BCAM (SEQ ID NO: 5) in tissue culture obtained from colorectal cancer patients.

### DETAILED DESCRIPTION OF THE INVENTION

Before the invention is described in detail with respect to some of its preferred embodiments, the following general definitions are provided.

The present invention as illustratively described in the following may suitably be practiced in the absence of any element or elements, limitation or limitations, not specifically disclosed herein.

The present invention will be described with respect to particular embodiments and with reference to certain figures but the invention is not limited thereto but only by the claims.

Where the term “comprising” is used in the present description and claims, it does not exclude other elements. For the purposes of the present invention, the term “consisting of” is considered to be a preferred embodiment of the term “comprising of”. If hereinafter a group is defined to comprise at least a certain number of embodiments, this is also to be understood to disclose a group which preferably consists only of these embodiments.

Where an indefinite or definite article is used when referring to a singular noun, e.g. “a”, “an” or “the”, this includes a plural of that noun unless something else is specifically stated. In the context of the present invention, the terms “about” or “approximately” denote an interval of accuracy that the person skilled in the art will understand to still ensure the technical effect of the feature in question. The term typically indicates deviation from the indicated numerical value of ±10%, and preferably of ±5%.

Further definitions of terms will be given in the following in the context of which the terms are used.

As mentioned in the background section, identification of molecular marker (patterns) for diagnosing CRC has focussed on the identification of genes the expression of which is either up- or down-regulated during CRC development. While such data provides valuable information, the diagnostic markers, i.e. genes and their products (e.g. polypeptides) may not all be suitable for non-invasive molecular imaging diagnostic approaches.

The inventors of the present invention have succeeded in identifying a set of polypeptides which are likely to be over-expressed in the majority of patients suffering from colorectal cancer development versus healthy individuals and which may be accessible for non-invasive molecular imaging diagnostic methods.

To this end, the inventors have devised a screening strategy comprising at least the following steps:

a) Obtaining cells from different human individuals all suffering from colorectal cancer and/or obtaining colorectal cancer cell lines;

b) Labeling polypeptides on the surface of said cells;

c) Isolating labelled polypeptides from non-labelled polypeptides;

d) Identification of isolated labelled polypeptides from the cell surface polypeptide-fraction of step c),

e) Of the polypeptides of step d), selecting those isolated labelled polypeptides for which genomic expression data indicate an increased expression compared to cells obtained from healthy individuals;

f) Of the polypeptides selected in step e), further selecting those isolated labelled polypeptides which comprise at least one transmembrane domain and/or at least one signal peptide;

g) Of the polypeptides selected in step f), further selecting those polypeptides with a positive RSC;

h) Of the polypeptides selected in step g), further selecting those polypeptides found within at least 70% of tested cells from different human individuals;

i) Of the polypeptides selected in step h), further selecting those polypeptides for which histological analysis confirms localization to the plasma membrane.

The cells of step a) which are obtained from human individuals suffering from CRC will typically be cell types that are known to be involved in the development of CRC. Typically, the cells will comprise embryonic, fetal or adult stem cells; progenitor cells; blood cells such as B and T-lymphocytes, monocytes and macrophages; epithelial cells; fibroblasts; and neuronal cells. Epithelial cells may be preferred.

The cells used in step a) may also be established CRC cell lines. Such cell lines are commercially available and include inter alia Colo205, HT-29, Caco-2, RKO, HCT116, SW1398, LS513, SW480, SW620 and COLO 320.

The labellng step b) is undertaken to allow for efficient purification, enrichment and isolation of preferentially polypeptides that localise to the surface of cells. Thus, one will use labels which allow for efficient purification, e.g. by affinity chromatography. Such labels may include e.g. biotin, maltose binding protein (MBP), Glutathione-S-transferase (GST), histidine-tags, Flag-tags, antibodies and the like.

The labels may be covalently or non-covalently attached to proteins being preferentially located on the cell surface.

For covalent modification one may use coupling chemistries that are commonly used for such purposes. Thus, one may use homo and/or hetero-bi and/or multifunctional crosslinking agents. Typical cross-linking agents include but are not limited to bis(sulfosuccinimid) bis(diazo-benzidine), Dimethyl Adipimidate, Dimethyl Pimelimidate, Dimethyl Suberaimidate, Disuccininnclyl Suberate, Glutaraldehyde, in-Maleimidobenzoyl-N-Hydroxysuccinimide, Sulfosuccinidyl 4-(N-Maleimidomethyl) Cyclohexane-1-carboxylate etc.

A preferred label may be sulfo-NHS-SS-biotin.

Subsequently, the cells are typically lysed. This may be achieved by hypotonic, enzymatic, ultrasound or mechanical cell rupture.

Subsequently, labelled polypeptides may be isolated making use of the label. The person skilled in the art knows how to select the appropriate isolation procedure.

If for example a biotin-label is used for labelling polypeptides, streptavidine-based chromatographic systems such as streptavidine-labelled or neuravidine-labelled beads as they are commercially available can be used. For His-tags, Ni-NTA agarose as available from Qiagen may be used. For Glutathion-S-transferase labels, GST-sepharose may be used etc.

By subjecting labelled polypeptides from e.g. lysed cells to such chromatographic media, purification of labelled polypeptides can be achieved. As the labelling procedure will typically preferentially label cell surface polypeptides, the fraction retained on and eluted from the chromatographic media may be designated as labelled cell surface polypeptide fraction. The polypeptides found in the flow through may be designated as the intracellular polypeptide fraction. Further, special preparation protocols as they are commonly known may be used to obtain fractions from the lysed cells comprising primarily cell surface associated polypeptides and intracellular polypeptides. Subjecting such fractions to chromatographic media as described above may improve adherence of labelled cell surface polypeptides to the chromatographic media.

If for example cells have been labelled with biotin, polypeptides of the cell will be preferentially labelled over cytoplasmic polypeptides such that labelled cell surface polypeptide will be preferentially retained on e.g. streptavidine-beads while cytoplasmic will be primarily found in the flow through or wash fractions.

The person skilled in the art is aware how to correctly perform these isolation/purification procedures, e.g. how to select suitable washing buffers, elution buffers, pH-values etc.

The labelled polypeptides from the cell surface polypeptide-fraction will thus be isolated by eluting them form the respective chromatographic media. Subsequently, such isolated labelled polypeptides will be identified. For this, one may use e.g. mass spectrometry (MS) analysis.

The data obtained by this procedure will then be correlated with genomic expression data on genes, the expression of which is up- or down-regulated in CRC. Genomic expression data may be obtained from e.g. Carvalho et al. (2009) Gut 58 (1), 79-89 which is incorporated by reference as far as it provides information on genes the expression of which is upregulated in CRC.

Correlation in the context of the present invention means that only those identified labelled polypeptides will be selected for which genomic expression data indicate an increased expression compared to cells obtained from healthy individuals.

In order to increase the likelihood that the labelled, isolated and identified polypeptides indeed localise to the plasma membrane of cells, a further selection criteria is applied which selects only those polypeptides for which an algorithm-based analysis indicates the presence of a transmembrane (TM) domain and/or at least one signal peptide. The term “signal peptide” refers to sequence signatures that direct localization of proteins to the plasma membrane of cells. Such signal peptide sequences are known in the art.

This analysis may be conducted using programmes and algorithms commonly used for such approaches such as Phobias developed by the Stockholm Bioinformatics Center (Käll et al. (2004) Journal of Molecular Biology, 338(5):1027-1036).

After selection of polypeptides with at least one transmembrane domain or at least one signal peptide, a further selection is applied to reduce the number of false positives. To this end, one selects only such proteins with a positive relative count (RSC) value.

In such an analysis, one determines which labelled polypeptides are enriched in the cell surface polypeptide fraction (i.e. preferentially retained on the chromatographic media) when compared to the intracellular polypeptide fraction. A log2 ratio measured from spectral counts (RSC) analysis is then performed. In principle, this analysis relies on a label-free quantification approach based on spectral counts and is suitable to calculate relative protein abundance between samples. A detailed description can be found in Old et al (2005) Molecular & Cellular Proteomics 4(10): 1487-1502 which is incorporated by reference as far as the determination of RSC values is concerned.

Using this analysis, only proteins with a positive RSC value are then selected.

The polypeptides identified by this series of different steps are then further analysed by identifying those polypeptides found within at least 70% of cells tested in step a) As pointed out above, the cells may be either obtained from different human individuals or may be established CRC cell lines. Typically, one will analyse at least five different cells meaning that e.g. at least cells from five different human individuals all suffering from CRC or at least five different CRC cell lines are analysed.

In a last selection step, polypeptides are selected for which histological analysis confirms localisation at least to the plasma membrane. Such histological analysis may be undertaken using e.g. antibodies being specific for the selected polypeptides.

Typically, the histological analysis will involve immunofluorescent analysis of cells obtained either from human individuals suffering from CRC or of established CRC cell lines. The specific protocols used for such histological analysis will usually depend on the specific polypeptide analysed. Nevertheless, the person skilled in the art will be able to rely on knowledge generally available for e.g. immunofluorescent detection and localisation of polypeptides.

Basically, such approaches will involve fixation of cells on a solid support such as a transparent microscopic cover slide. Subsequently, the cells are lysed and fixed before labelling with e.g. antibodies is performed.

Such approaches are described inter alia in Carvalho et al. (2009) Gut 58 (1), 79-89.

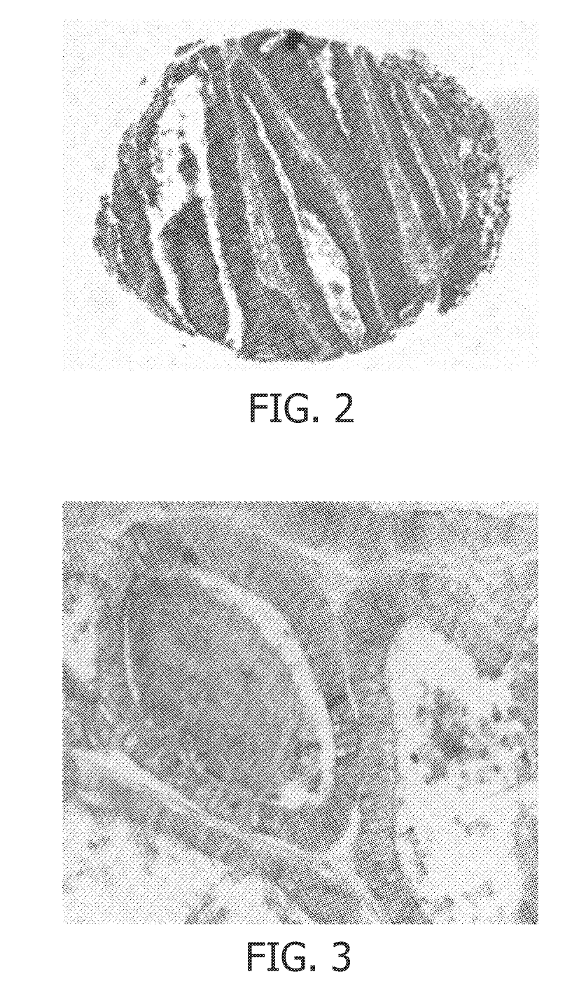

The inventors of the present invention relying on the steps a) to step h) have succeeded in identifying a set of 32 proteins which fulfil the aforementioned criteria. Further, histological analysis of PRNP (SEQ ID NO: 6) and BCAM (SEQ ID NO: 5) confirmed that these two proteins indeed localise at least to the plasma membrane (see FIG. 2 and FIG. 3, respectively).

A summary of the properties of these 32 proteins can be taken from Table 1.

TABLE 1

| | SEQ ID | TM | Protein | cell line | | DIFF IN |
|---|---|---|---|---|---|---|
| Protein name | No: | region | MW | count | RSC | EXPRESSION |
| PLXNA1 Plexin-A1 precursor | 1 | 1 | 211071.9 | 5 | 5.972006932 | −0.616236226 |
| SLC1A5 Neutral amino acid transporter B | 2 | 9 | 56581.8 | 5 | 2.653686031 | −0.292097734 |
| SCARB1 Isoform 1 of Scavenger receptor class B member 1 | 3 | 1 | 56956.6 | 5 | 4.256484988 | −0.139340706 |
| ICAM1 Intercellular adhesion molecule 1 precursor | 4 | 1 | 57806.5 | 5 | 4.688794738 | −0.502549651 |
| BCAM Lutheran blood group glycoprotein precursor | 5 | 1 | 67385.9 | 5 | 6.546313113 | −0.511112343 |
| PRNP Major prion protein precursor | 6 | 2 | 27642.7 | 5 | 4.756670474 | −0.549604991 |
| DAG1 Dystroglycan precursor | 7 | 1 | 97563.3 | 5 | 6.54021969 | −0.248052408 |
| PTGFRN Prostaglandin F2 receptor negative regulator precursor | 8.32 | 1 | 98537.5 | 4 | 6.027860921 | −0.298927655 |
| IGSF8 Isoform 1 of Immunoglobulin superfamily member 8 precursor | 9 | 1 | 65014.8 | 5 | 4.376764152 | −0.32307837 |
| LDLR Low-density lipoprotein receptor precursor | 10 | 1 | 95356.7 | 5 | 5.046917378 | −0.409453599 |
| SLC1A3 Excitatory amino acid transporter 1 | 11 | 7 | 59555.8 | 4 | 3.673690871 | −0.56633461 |
| ACSL1 Isoform 1 of Long-chain-fatty-acid--CoA ligase 1 | 12 | 1 | 77927.1 | 4 | 2.494870467 | −0.468910389 |
| ITFG3 69 kDa protein | 13 | 1 | 69329.2 | 5 | 3.369073363 | −0.537911898 |
| LRP8 Isoform 1 of Low-density lipoprotein receptor-related protein 8 precursor | 14 | 1 | 105658.4 | 4 | 1.495012337 | −0.447612397 |
| CYB5B cytochrome b5 outer mitochondrial membrane precursor | 15 | 1 | 16676.8 | 5 | 3.31042916 | −0.40857396 |
| COPS6 COP9 signalosome complex subunit 6 | 16 | 1 | 36145.4 | 5 | 1.0369939 | −0.243432062 |
| PRSS8 Prostasin precursor | 17 | 2 | 36745.6 | 4 | 4.462248084 | −0.603233346 |
| SDC4 Syndecan-4 precursor | 18 | 1 | 21624.1 | 4 | 2.331824104 | −0.636360718 |
| SLC2A1 Solute carrier family 2, facilitated glucose transporter member 1 | 19 | 12 | 54067.3 | 5 | 2.144242719 | −0.65733993 |
| BST2 Bone marrow stromal antigen 2 precursor | 20 | 1 | 19751.3 | 4 | 3.045513157 | −0.902767067 |
| HSD17B7 Isoform 1 of 3-keto-steroid reductase | 21 | 1 | 38189.9 | 5 | 2.505469898 | −0.19805186 |
| CPOX Coproporphyrinogen III oxidase, mitochondrial precursor | 22 | 1 | 50133.9 | 5 | 1.673454923 | −0.22411626 |
| PODXL Podocalyxin-like protein 1 precursor | 23 | 1 | 55577.7 | 4 | 2.807508469 | −0.376649598 |
| DHCR7 7-dehydrocholesterol reductase | 24 | 7 | 54473 | 5 | 1.858119548 | −0.350599051 |
| SLC4A2 Isoform A of Anion exchange protein 2 | 25 | 13 | 136994.4 | 4 | 4.5335838 | −0.284851412 |
| CNNM3 cyclin M3 isoform 1 | 26 | 3 | 76102.5 | 4 | 2.904753941 | −0.126520099 |
| SLC7A11 Cystine/glutamate transporter | 27 | 14 | 55407.5 | 5 | 3.275370303 | −0.20939536 |
| KIAA1524 Isoform 1 of Protein KIAA1524 | 28 | 1 | 102171.3 | 4 | 2.419056717 | −0.281066707 |
| AUP1 Isoform Long of Ancient ubiquitous protein 1 precursor | 29 | 2 | 53010.8 | 4 | 2.526552401 | −0.187455693 |
| SLC1A4 Neutral amino acid transporter A | 30 | 9 | 55706.4 | 4 | 2.369591427 | −0.186930822 |
| TMEM16F Transmembrane protein 16F | 31 | 8 | 106151.4 | 5 | 3.310893761 | −0.205564206 |

“Tm” indicates the number of transmembrane domains.
“Protein MW” indicates the theoretic molecular weight.
“Cell line count” indicates in how many of the five CRC cell lines Colo205, Caco2, HT29, RKO and HCT 116 the respective polypeptide was observed.
“RSC” indicates the RSC value measured.
“DIFF IN EXPRESSION” indicates the difference by which the gene is overexpressed in CRC based on the data of Carvalho et al. (2009) Gut 58 (1).

By applying even stricter selection criteria (namely using an RSC of at least 2.5, a set of 11 proteins emerged which may be particularly useful as diagnostic markers, or targets for contrast agents. This list of 11 polypeptides is shown in Table 2.

TABLE 2

| | | TM | Protein | cell line | | DIFF |
|---|---|---|---|---|---|---|
| Protein name | SEQ ID No: | region | MW | count | RSC | IN EXPRESSION |
| PLXNA1 Plexin-A1 precursor | 1 | 1 | 211071.9 | 5 | 5.972006932 | −0.616236226 |
| SLC1A5 Neutral amino acid transporter B | 2 | 9 | 56581.8 | 5 | 2.653686031 | −0.292097734 |
| SCARB1 Isoform 1 of Scavenger receptor class B member 1 | 3 | 1 | 56956.6 | 5 | 4.256484988 | −0.139340706 |
| ICAM1 Intercellular adhesion molecule 1 precursor | 4 | 1 | 57806.5 | 5 | 4.688794738 | −0.502549651 |
| BCAM Lutheran blood group glycoprotein precursor | 5 | 1 | 67385.9 | 5 | 6.546313113 | −0.511112343 |
| PRNP Major prion protein precursor | 6 | 2 | 27642.7 | 5 | 4.756670474 | −0.549604991 |
| DAG1 Dystroglycan precursor | 7 | 1 | 97563.3 | 5 | 6.54021969 | −0.248052408 |
| PTGFRN Prostaglandin F2 receptor negative regulator precursor | 8.32 | 1 | 98537.5 | 4 | 6.027860921 | −0.298927655 |
| IGSF8 Isoform 1 of Immunoglobulin superfamily member 8 precursor | 9 | 1 | 65014.8 | 5 | 4.376764152 | −0.32307837 |
| LDLR Low-density lipoprotein receptor precursor | 10 | 1 | 95356.7 | 5 | 5.046917378 | −0.409453599 |

Tm” indicates the number of transmembrane domains.
“Protein MW” indicates the theoretic molecular weight.
“Cell line count” indicates in how many of the five CRC cell lines Colo205, Caco2, HT29, RKO and HCT 116 the respective polypeptide was observed.
“RSC” indicates the RSC value measured.
“DIFF IN EXPRESSION” indicates the difference by which the gene is overexpressed in CRC based on the data of Carvalho et al. (2009) Gut 58 (1) (Is this correct? YES).

Accordingly, one embodiment of the present invention relates to a diagnostic marker, preferably for detecting CRC comprising at least one polypeptide of the group consisting of:
A polypeptide of SEQ ID No.: 1;
A polypeptide of SEQ ID No.: 2;
A polypeptide of SEQ ID No.: 3;
A polypeptide of SEQ ID No.: 4;
A polypeptide of SEQ ID No.: 5;
A polypeptide of SEQ ID No.: 6;
A polypeptide of SEQ ID No.: 7;
A polypeptide of SEQ ID No.: 8;
A polypeptide of SEQ ID No.: 9;
A polypeptide of SEQ ID No.: 10;
A polypeptide of SEQ ID No.: 11;
A polypeptide of SEQ ID No.: 12;
A polypeptide of SEQ ID No.: 13;
A polypeptide of SEQ ID No.: 14;
A polypeptide of SEQ ID No.: 15;
A polypeptide of SEQ ID No.: 16;
A polypeptide of SEQ ID No.: 17;
A polypeptide of SEQ ID No.: 18;
A polypeptide of SEQ ID No.: 19;
A polypeptide of SEQ ID No.: 20;
A polypeptide of SEQ ID No.: 21;
A polypeptide of SEQ ID No.: 22;
A polypeptide of SEQ ID No.: 23;
A polypeptide of SEQ ID No.: 24;
A polypeptide of SEQ ID No.: 25;
A polypeptide of SEQ ID No.: 26;
A polypeptide of SEQ ID No.: 27;
A polypeptide of SEQ ID No.: 28;
A polypeptide of SEQ ID No.: 29;
A polypeptide of SEQ ID No.: 30;
A polypeptide of SEQ ID No.: 31; and/or
A polypeptide of SEQ ID No.: 32.

The term “diagnostic marker” refers to the property of polypeptides of SEQ ID No. 1 to 32 as being suitable as a marker for detecting cancers such as CRC development. Thus, detection of at least one polypeptide of SEQ ID No. 1 to SEQ ID No. 32 will, in principle, allow one to decide whether a human individual for which over-expression of such a polypeptide in comparison to a suitable control has been shown suffers from e.g. CRC.

The person skilled in the art will be aware that the simultaneous detection of more than at least one polypeptide of SEQ ID No. 1 to 32 will increase the correctness of the prediction for the occurrence of e.g. CRC.

The present invention therefore also relates in a preferred embodiment to a diagnostic marker or a set of diagnostic markers comprising at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least twenty one, at least twenty two, at least twenty three, at least twenty four, at least twenty five, at least twenty six, at least twenty seven, at least twenty eight, at least twenty nine, at least thirty or thirty one polypeptides of the group consisting of SEQ ID Nos. 1 to 32.

A preferred diagnostic marker in accordance with the present invention relates to a diagnostic marker or a set of diagnostic markers comprising at least ten polypeptides of the group consisting of SEQ ID No. 1 to 10 and SEQ ID No. 32.

A further preferred diagnostic marker relates to a diagnostic marker or a set of diagnostic markers comprising at least the polypeptides of SEQ ID Nos. 1, 2 and 3.

Yet another preferred diagnostic marker of the present invention relates to a diagnostic marker or a set of diagnostic markers comprising at least the polypeptides of SEQ ID No. 1, 2, 3 and 4.

The present invention also relates to the use of at least one polypeptide selected from the group consisting of:
A polypeptide of SEQ ID No.: 1;
A polypeptide of SEQ ID No.: 2;
A polypeptide of SEQ ID No.: 3;
A polypeptide of SEQ ID No.: 4;
A polypeptide of SEQ ID No.: 5;
A polypeptide of SEQ ID No.: 6;
A polypeptide of SEQ ID No.: 7;
A polypeptide of SEQ ID No.: 8;
A polypeptide of SEQ ID No.: 9;
A polypeptide of SEQ ID No.: 10;
A polypeptide of SEQ ID No.: 11;
A polypeptide of SEQ ID No.: 12;
A polypeptide of SEQ ID No.: 13;
A polypeptide of SEQ ID No.: 14;
A polypeptide of SEQ ID No.: 15;
A polypeptide of SEQ ID No.: 16;
A polypeptide of SEQ ID No.: 17;
A polypeptide of SEQ ID No.: 18;
A polypeptide of SEQ ID No.: 19;
A polypeptide of SEQ ID No.: 20;
A polypeptide of SEQ ID No.: 21;
A polypeptide of SEQ ID No.: 22;
A polypeptide of SEQ ID No.: 23;
A polypeptide of SEQ ID No.: 24;
A polypeptide of SEQ ID No.: 25;
A polypeptide of SEQ ID No.: 26;
A polypeptide of SEQ ID No.: 27;
A polypeptide of SEQ ID No.: 28;
A polypeptide of SEQ ID No.: 29;
A polypeptide of SEQ ID No.: 30;
A polypeptide of SEQ ID No.: 31; and/or
A polypeptide of SEQ ID No.: 32;
   as a diagnostic marker for detecting CRC.

The present invention also relates to the use of at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least twenty one, at least twenty two, at least twenty three, at least twenty four, at least twenty five, at least twenty six, at least twenty seven, at least twenty eight, at least twenty nine, at least thirty or thirty one polypeptides of the group consisting of SEQ ID Nos. 1 to 32 as diagnostic markers for detecting CRC.

Preferably the invention relates to the use of at least ten polypeptides of the group consisting of SEQ ID No. 1 to 10 and SEQ ID No.: 32 as a diagnostic marker for detecting CRC.

Another preferred embodiment relates to the use of the polypeptides of SEQ ID Nos. 1, 2 and 3 as a diagnostic marker for detecting CRC.

Yet another preferred embodiment relates to the use of the polypeptides of SEQ ID Nos. 1, 2, 3 and 4 as a diagnostic marker for detecting CRC.

In a preferred embodiment, the polypeptides selected from the group consisting of SEQ ID No. 1 to SEQ ID No. 32 and the above mentioned preferred combinations of polypeptides such as e.g. polypeptides with SEQ ID Nos. 1 to 10 and SEQ ID No.: 32, polypeptides of SEQ ID Nos. 1, 2 and 3, or polypeptides of SEQ ID Nos. 1, 2, 3 and 4 will be used in a diagnostic approach outside the human or animal body.

Another embodiment of the present invention uses at least one polypeptide selected from the group consisting of SEQ ID Nos: 1 to 32 or at least the aforementioned preferred combinations in a diagnostic approach that allows for online detection of the diagnostic marker within a human individual. Such a diagnostic approach may rely on magnetic resonance imaging (MRI) and/or magnetic photon resonance imaging (MPI). However, other approaches which allow the imaging of the presence of at least one polypeptide selected from the group consisting of SEQ ID Nos. 1 to 32 in order to detect CRC may also be applied.

Thus, in one embodiment the present invention relates to a contrast agent being capable of specifically detecting at least polypeptide selected from the group consisting of SEQ ID Nos. 1 to 32.

The term “contrast agent” and its grammatical variations refers to a molecular compound that is capable of specifically interacting with a polypeptide of SEQ ID Nos. 1 to 32 and which can be detected by an apparatus positioned outside the human or animal body. Preferably, such contrast agents are suitable for use in magnetic resonance imaging (MRI) or magnetic photon imaging (MPI).

The term “specifically interacting” and its grammatical variations refer to the property of a molecular compound to preferentially interact with a polypeptide of SEQ ID Nos. 1 to 32 on the cell surface of cells being present within the human or animal body over other proteins that are expressed by such cells.

Preferred contrast agents which may also be designated as contrast agent compositions will be capable of specifically detecting at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least twenty one, at least twenty two, at least twenty three, at least twenty four, at least twenty five, at least twenty six, at least twenty seven, at least twenty eight, at least twenty nine, at least thirty or thirty one polypeptides of the group consisting of SEQ ID Nos. 1 to 32. Other preferred contrast agents/compositions will be capable of detecting the group of polypeptides consisting of SEQ ID No.: 1 to 10 and SEQ ID No.: 32, the group of SEQ ID Nos. 1, 2 and 3 or the group of SEQ ID Nos. 1, 2, 3 and 4. As MRI and MPI in principle allow for non-invasive molecular imaging, contrast agents which comprise compounds that are capable of specifically interacting with at least one polypeptide of SEQ ID Nos. 1 to 32 and preferably with the aforementioned groups and which can be detected by MRI and/or MPI are preferred.

A class of molecules which may be particularly suitable as contrast agents for the purposes of the present invention are antibodies.

Antibodies being specific for polypeptides of SEQ ID Nos. 1 to 32 and preferably for the aforementioned groups of polypeptides may be already commercially available such as Primary anti-prion, mouse clone 8H4 antibody from Sigma-Aldrich (St. Louis, Mo., USA) (for SEQ ID No.6), mouse anti human CD239 from abD serotech (Oxford, UK) (for SEQ ID No. 5).

If antibodies specifically recognising polypeptides of SEQ ID Nos. 1 to 32 are not already available, they can be produced by methods known to the person skilled in the art. Such antibodies may be polyclonal or monoclonal antibodies. Monoclonal antibodies may be produced by classical hybridoma fusion technology or e.g. phage-display systems.

The term “antibody” is used in the context of the present invention in its common sense. However, in addition the term further includes antibody variants and derivatives such as a single chain antibody, a Fab-fragment, a Fab2-fragment, a multispecific antibody, a diabody, a triabody, a tetrabody, a minibody, a linear antibody, a chelating recombinant antibody, a tribody, a bibody, an intrabody, a nanobody, a small modular immunopharmaceutical (SMIP), a binding-domain immunoglobulin fusion protein, a camelized antibody, a V_{HH} containing antibody and the like.

Preferred embodiments of the present invention relate to contrast agents which comprise antibodies that interact at least with polypeptides of SEQ ID Nos. 1 to 10 and SEQ ID No.: 32, with polypeptides of SEQ ID Nos. 1, 2 and 3 or with polypeptides of SEQ ID Nos. 1, 2, 3 and 4.

Contrast agents comprising such antibodies may be provided in a form where the contrast agent is a composition comprising different sets of antibody with each antibody recognising a polypeptide of a specific SEQ ID. Thus, a contrast agent may comprise three antibodies recognising polypeptides of SEQ ID Nos. 1, 2 and 3, respectively.

However, contrast agents in accordance with the present invention may also comprise antibodies which are multi-specific. Thus, a contrast agent may comprise a single antibody which recognises e.g. the polypeptide of SEQ ID No. 1, 2 and 3, at the same time. Further, contrast agents in accordance with the present invention may comprise antibodies all of which recognise a polypeptide of the same SEQ ID. Thus, a contrast agent in accordance with the present invention may comprise e.g. three antibodies all of which are specific for SEQ ID No. 1 and e.g. four antibodies all of which are specific for SEQ ID No. 2 etc.

Contrast agents, aside from their property of being capable of specifically recognising polypeptides of SEQ ID Nos. 1 to 32 will in addition typically comprise a marker molecule which is detectable by the specific detection technology used.

For example, if fluorescent spectroscopy is used as a detection means, such marker molecules may comprise fluorophores as detectable marker molecules that can be excitated at a specific wavelength.

With respect to preferred contrast agents in accordance with the invention that are suitable for MRI, the contrast agents such as the above described antibodies may comprise a marker molecule which is detectable by MRI. Such detectable labels include e.g. USPIOS and 19-Fluor.

Another embodiment of the present invention relates to the use of at least one antibody capable of interacting with a polypeptide selected from the group consisting of:
A polypeptide of SEQ ID No.: 1;
A polypeptide of SEQ ID No.: 2;
A polypeptide of SEQ ID No.: 3;
A polypeptide of SEQ ID No.: 4;
A polypeptide of SEQ ID No.: 5;
A polypeptide of SEQ ID No.: 6;
A polypeptide of SEQ ID No.: 7;
A polypeptide of SEQ ID No.: 8;
A polypeptide of SEQ ID No.: 9;
A polypeptide of SEQ ID No.: 10;
A polypeptide of SEQ ID No.: 11;
A polypeptide of SEQ ID No.: 12;
A polypeptide of SEQ ID No.: 13;
A polypeptide of SEQ ID No.: 14;
A polypeptide of SEQ ID No.: 15;
A polypeptide of SEQ ID No.: 16;
A polypeptide of SEQ ID No.: 17;
A polypeptide of SEQ ID No.: 18;
A polypeptide of SEQ ID No.: 19;
A polypeptide of SEQ ID No.: 20;
A polypeptide of SEQ ID No.: 21;
A polypeptide of SEQ ID No.: 22;
A polypeptide of SEQ ID No.: 23;
A polypeptide of SEQ ID No.: 24;
A polypeptide of SEQ ID No.: 25;
A polypeptide of SEQ ID No.: 26;
A polypeptide of SEQ ID No.: 27;
A polypeptide of SEQ ID No.: 28;
A polypeptide of SEQ ID No.: 29;
A polypeptide of SEQ ID No.: 30;
A polypeptide of SEQ ID No.: 31; and/or
A polypeptide of SEQ ID No.: 32
   as a contrast agent suitable for MRI.

The invention also relates to the use of antibodies which interact with at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least twenty one, at least twenty two, at least twenty three, at least twenty four, at least twenty five, at least twenty six, at least twenty seven, at least twenty eight, at least twenty nine, at least thirty or thirty one polypeptides of the group consisting of SEQ ID Nos. 1 to 32 as contrast agents.

Preferably the invention relates to the use of antibodies which interact with at least ten polypeptides of the group consisting of SEQ ID No. 1 to 10 and SEQ ID No.: 32 as contrast agents.

Another preferred embodiment relates to the use of antibodies which interact with the polypeptides of SEQ ID Nos. 1, 2 and 3 as contrast agents.

Yet another preferred embodiment relates to the use of antibodies which interact with the polypeptides of SEQ ID Nos. 1, 2, 3 and 4 as contrast agents.

As pointed out above, the diagnostic markers and contrast agents of the present invention may be used for detection and diagnosis of colorectal cancer. These methods may be used either outside or inside the human or animal body. Thus, one may e.g. use an antibody which is labelled to a fluorophore in the histological analysis for detecting polypeptides of SEQ ID Nos. 1 to 32 in cellular tissue and samples which have been obtained from an individual suspected of suffering from CRC.

In addition or alternatively, one may use e.g. antibodies for non-invasive molecular imaging techniques such as MRI.

As the diagnostic markers and contrast agents as described above are primarily used for the detection and diagnosis of CRC, one embodiment of the present invention relates to a method of diagnosing CRC comprising at least the following steps:

a) Obtaining at least one sample from at least one human or animal individual suspected to suffer from ongoing or imminent CRC development;

b) Testing in said at least one sample for expression of at least one polypeptide selected from the group consisting of:
A polypeptide of SEQ ID No.: 1;
A polypeptide of SEQ ID No.: 2;
A polypeptide of SEQ ID No.: 3;
A polypeptide of SEQ ID No.: 4;
A polypeptide of SEQ ID No.: 5;
A polypeptide of SEQ ID No.: 6;
A polypeptide of SEQ ID No.: 7;
A polypeptide of SEQ ID No.: 8;
A polypeptide of SEQ ID No.: 9;
A polypeptide of SEQ ID No.: 10;
A polypeptide of SEQ ID No.: 11;
A polypeptide of SEQ ID No.: 12;
A polypeptide of SEQ ID No.: 13;
A polypeptide of SEQ ID No.: 14;
A polypeptide of SEQ ID No.: 15;
A polypeptide of SEQ ID No.: 16;
A polypeptide of SEQ ID No.: 17;
A polypeptide of SEQ ID No.: 18;
A polypeptide of SEQ ID No.: 19;
A polypeptide of SEQ ID No.: 20;
A polypeptide of SEQ ID No.: 21;
A polypeptide of SEQ ID No.: 22;
A polypeptide of SEQ ID No.: 23;
A polypeptide of SEQ ID No.: 24;
A polypeptide of SEQ ID No.: 25;
A polypeptide of SEQ ID No.: 26;
A polypeptide of SEQ ID No.: 27;
A polypeptide of SEQ ID No.: 28;
A polypeptide of SEQ ID No.: 29;
A polypeptide of SEQ ID No.: 30;
A polypeptide of SEQ ID No.: 31; and/or
A polypeptide of SEQ ID No.: 32;

c) Testing in at least one control sample obtained from at least one human or animal individual not suffering from ongoing or imminent CRC development for expression of at least one polypeptide selected from the group consisting of:
A polypeptide of SEQ ID No.: 1;
A polypeptide of SEQ ID No.: 2;
A polypeptide of SEQ ID No.: 3;
A polypeptide of SEQ ID No.: 4;
A polypeptide of SEQ ID No.: 5;
A polypeptide of SEQ ID No.: 6;
A polypeptide of SEQ ID No.: 7;
A polypeptide of SEQ ID No.: 8;
A polypeptide of SEQ ID No.: 9;
A polypeptide of SEQ ID No.: 10;
A polypeptide of SEQ ID No.: 11;
A polypeptide of SEQ ID No.: 12;
A polypeptide of SEQ ID No.: 13;
A polypeptide of SEQ ID No.: 14;
A polypeptide of SEQ ID No.: 15;
A polypeptide of SEQ ID No.: 16;
A polypeptide of SEQ ID No.: 17;
A polypeptide of SEQ ID No.: 18;
A polypeptide of SEQ ID No.: 19;
A polypeptide of SEQ ID No.: 20;
A polypeptide of SEQ ID No.: 21;
A polypeptide of SEQ ID No.: 22;
A polypeptide of SEQ ID No.: 23;
A polypeptide of SEQ ID No.: 24;
A polypeptide of SEQ ID No.: 25;
A polypeptide of SEQ ID No.: 26;
A polypeptide of SEQ ID No.: 27;
A polypeptide of SEQ ID No.: 28;
A polypeptide of SEQ ID No.: 29;
A polypeptide of SEQ ID No.: 30;
A polypeptide of SEQ ID No.: 31
A polypeptide of SEQ ID No.: 32;

d) Determining difference in expression of steps b) and d);

e) Deciding on the presence or imminence of CRC development based on the results obtained in step d).

In one embodiment, steps b), c), d) and/or e) of this method of diagnosis are performed outside the human or animal body.

Another embodiment of the present invention relates to a method of diagnosing CRC comprising at least the following steps:

a) Testing in at least one human or animal individual suspected to suffer from ongoing or imminent CRC development for expression of at least one polypeptide selected from the group consisting of:
A polypeptide of SEQ ID No.: 1;
A polypeptide of SEQ ID No.: 2;
A polypeptide of SEQ ID No.: 3;
A polypeptide of SEQ ID No.: 4;
A polypeptide of SEQ ID No.: 5;
A polypeptide of SEQ ID No.: 6;
A polypeptide of SEQ ID No.: 7;
A polypeptide of SEQ ID No.: 8;
A polypeptide of SEQ ID No.: 9;
A polypeptide of SEQ ID No.: 10;
A polypeptide of SEQ ID No.: 11;
A polypeptide of SEQ ID No.: 12;
A polypeptide of SEQ ID No.: 13;
A polypeptide of SEQ ID No.: 14;
A polypeptide of SEQ ID No.: 15;
A polypeptide of SEQ ID No.: 16;
A polypeptide of SEQ ID No.: 17;
A polypeptide of SEQ ID No.: 18;
A polypeptide of SEQ ID No.: 19;
A polypeptide of SEQ ID No.: 20;
A polypeptide of SEQ ID No.: 21;
A polypeptide of SEQ ID No.: 22;
A polypeptide of SEQ ID No.: 23;
A polypeptide of SEQ ID No.: 24;
A polypeptide of SEQ ID No.: 25;
A polypeptide of SEQ ID No.: 26;
A polypeptide of SEQ ID No.: 27;
A polypeptide of SEQ ID No.: 28;
A polypeptide of SEQ ID No.: 29;
A polypeptide of SEQ ID No.: 30;
A polypeptide of SEQ ID No.: 31
A polypeptide of SEQ ID No.: 32;

b) Comparing expression as determined in step a) with expression of at least one polypeptide selected from the group consisting of:
A polypeptide of SEQ ID No.: 1;
A polypeptide of SEQ ID No.: 2;
A polypeptide of SEQ ID No.: 3;
A polypeptide of SEQ ID No.: 4;
A polypeptide of SEQ ID No.: 5;
A polypeptide of SEQ ID No.: 6;
A polypeptide of SEQ ID No.: 7;
A polypeptide of SEQ ID No.: 8;
A polypeptide of SEQ ID No.: 9;
A polypeptide of SEQ ID No.: 10;
A polypeptide of SEQ ID No.: 11;
A polypeptide of SEQ ID No.: 12;
A polypeptide of SEQ ID No.: 13;
A polypeptide of SEQ ID No.: 14;
A polypeptide of SEQ ID No.: 15;
A polypeptide of SEQ ID No.: 16;
A polypeptide of SEQ ID No.: 17;
A polypeptide of SEQ ID No.: 18;
A polypeptide of SEQ ID No.: 19;
A polypeptide of SEQ ID No.: 20;
A polypeptide of SEQ ID No.: 21;
A polypeptide of SEQ ID No.: 22;
A polypeptide of SEQ ID No.: 23;
A polypeptide of SEQ ID No.: 24;
A polypeptide of SEQ ID No.: 25;
A polypeptide of SEQ ID No.: 26;
A polypeptide of SEQ ID No.: 27;
A polypeptide of SEQ ID No.: 28;
A polypeptide of SEQ ID No.: 29;
A polypeptide of SEQ ID No.: 30;
A polypeptide of SEQ ID No.: 31; and/or
A polypeptide of SEQ ID No.: 32;
   as determined for human or animal individuals not suffering from ongoing or imminent colorectal cancer development.

c) Deciding on the presence or imminence of colorectal cancer development based on the results obtained in step b).

Further, the present invention may relate to a method of data acquisition comprising at least the following steps:

a) Testing in at least one human or animal individual suspected to suffer from ongoing or imminent CRC development for expression of at least one polypeptide selected from the group consisting of:
A polypeptide of SEQ ID No.: 1;
A polypeptide of SEQ ID No.: 2;
A polypeptide of SEQ ID No.: 3;
A polypeptide of SEQ ID No.: 4;
A polypeptide of SEQ ID No.: 5;
A polypeptide of SEQ ID No.: 6;
A polypeptide of SEQ ID No.: 7;
A polypeptide of SEQ ID No.: 8;
A polypeptide of SEQ ID No.: 9;
A polypeptide of SEQ ID No.: 10;
A polypeptide of SEQ ID No.: 11;
A polypeptide of SEQ ID No.: 12;
A polypeptide of SEQ ID No.: 13;
A polypeptide of SEQ ID No.: 14;
A polypeptide of SEQ ID No.: 15;
A polypeptide of SEQ ID No.: 16;
A polypeptide of SEQ ID No.: 17;
A polypeptide of SEQ ID No.: 18;
A polypeptide of SEQ ID No.: 19;
A polypeptide of SEQ ID No.: 20;
A polypeptide of SEQ ID No.: 21;
A polypeptide of SEQ ID No.: 22;
A polypeptide of SEQ ID No.: 23;
A polypeptide of SEQ ID No.: 24;
A polypeptide of SEQ ID No.: 25;
A polypeptide of SEQ ID No.: 26;
A polypeptide of SEQ ID No.: 27;
A polypeptide of SEQ ID No.: 28;
A polypeptide of SEQ ID No.: 29;
A polypeptide of SEQ ID No.: 30;
A polypeptide of SEQ ID No.: 31; and/or
A polypeptide of SEQ ID No.: 32;

b) Comparing expression as determined in step a) with expression of at least one polypeptide selected from the group consisting of:
A polypeptide of SEQ ID No.: 1;
A polypeptide of SEQ ID No.: 2;
A polypeptide of SEQ ID No.: 3;
A polypeptide of SEQ ID No.: 4;
A polypeptide of SEQ ID No.: 5;
A polypeptide of SEQ ID No.: 6;
A polypeptide of SEQ ID No.: 7;
A polypeptide of SEQ ID No.: 8;
A polypeptide of SEQ ID No.: 9;
A polypeptide of SEQ ID No.: 10;
A polypeptide of SEQ ID No.: 11;
A polypeptide of SEQ ID No.: 12;
A polypeptide of SEQ ID No.: 13;
A polypeptide of SEQ ID No.: 14;
A polypeptide of SEQ ID No.: 15;
A polypeptide of SEQ ID No.: 16;
A polypeptide of SEQ ID No.: 17;
A polypeptide of SEQ ID No.: 18;
A polypeptide of SEQ ID No.: 19;
A polypeptide of SEQ ID No.: 20;
A polypeptide of SEQ ID No.: 21;
A polypeptide of SEQ ID No.: 22;
A polypeptide of SEQ ID No.: 23;
A polypeptide of SEQ ID No.: 24;
A polypeptide of SEQ ID No.: 25;
A polypeptide of SEQ ID No.: 26;
A polypeptide of SEQ ID No.: 27;
A polypeptide of SEQ ID No.: 28;
A polypeptide of SEQ ID No.: 29;
A polypeptide of SEQ ID No.: 30;
A polypeptide of SEQ ID No.: 31; and/or
A polypeptide of SEQ ID No.: 32;
   as determined for human or animal individuals not suffering from ongoing or imminent CRC development.

The afore-described methods have in common that they determine the occurrence of at least one polypeptide of SEQ ID Nos. 1 to 32 either in test samples obtained from human or animal individuals being suspected of suffering from CRC development or directly in human or animal individuals being suspected of suffering from CRC development.

Further, the data obtained for these molecules are then compared with the data obtained either from control samples or control individuals. The comparison with the control samples or control individuals serves to identify these test samples obtained from human individuals being suspected of suffering from CRC or to identify these human individuals being suspected of suffering from CRC in which at least one polypeptide of SEQ ID Nos. 1 to 32 is over-expressed compared to the respective control sample or control individual.

The term “control sample” or “control individual” therefore refers either to a sample obtained from e.g. a human individual or to e.g. a human individual not suffering from CRC cancer. Such individuals may be identified by performing classical CRC diagnosis. A person skilled in the art will be aware that for the purposes of the comparison between a test sample versus control sample and a test individual versus control individual, a proper standardization of the obtained expression data must be undertaken. However, such standardization is common in the art and usually includes determination of expression of polypeptides of SEQ ID Nos. 1 to 32 in relation to e.g. a polypeptide not being involved in colorectal cancer. Thus, in one aspect, data obtained from test samples and control samples may be standardized with respect to expression of a compound such as e.g. actine.

In preferred embodiments of the above mentioned methods of diagnosis or data acquisition, one monitors expression of at least the ten polypeptides of SEQ ID No.1 to 10 and SEQ ID No.: 32, of the polypeptides of SEQ ID No. 1, 2, 3 and 4, or of the polypeptides of SEQ ID No. 1, 2 and 3.

In the following, the present invention will be described with respect to some specific examples. These examples are however not to be construed as being limiting.

### EXAMPLES

### Experiment 1

### Identification of Cell Surface Proteins Potentially Involved in Colorectal Cancer

Colorectal cancer (CRC) cell lines COLO 205, HT-29, Caco-2, RKO and HCT116 were obtained from the American Type Culture Collection (ATCC). These are all cell lines used as model system for CRC (Lengauer. et al (1997) PNAS 94(6):2545-2550).

All cells except CACO2 were grown in complete medium (Dulbecco's Modified Eagle's Medium, DMEM; Lonza Biowhittaker, Verviers, Belgium) containing 10% Fetal Calf Serum (FCS) and 1% Penicillin/Streptomycin (penicillin 50 units/ml, Astellas Pharma B.V., Leiderdorp, Netherlands; streptomycin 50 ug/ml, Fisiopharma, Palomonta (SA), Italy) at 37° C. with a CO₂ atmosphere concentration of 5%. The CACO2 cell line was grown in RPMI1640 (Lonza Biowhittaker, Verviers, Belgium) containing 20% of FCS and 1% Penicillin/Streptomycin (penicillin 50 units/ml, Astellas Pharma B.V., Leiderdorp, Netherlands); streptomycin 50 ug/ml (Fisiopharma, Palomonta (SA), Italy) at 37° C. with a CO₂ atmosphere concentration of 5%.

The cell lines were grown until a confluency of 70/80% was obtained.

Subsequently, the cells were labelled with biotin by incubating the cells at 4° C. for 30 min with 412 μM Sulfo-NHS-SS-Biotin (Pierce, Rockford, USA) dissolved in PBS.

Following this incubation the cells were washed with PBS and lysed in lysis buffer (Pierce) supplemented with protease inhibitor cocktail (pic) (Complete Protease Inhibitor Cocktail, 1x, Roche, Mannheim, Germany)

The cell lysate was incubated at 4° C. for 120 min with NeutrAvidin Protein beads (Pierce) in a rotator to achieve binding of the biotin labelled proteins to the beads. The beads were than washed in two wash buffers. First three times in wash buffer A containing 1% w/v nonidet P-40 and 0.1% w/v SDS in PBS followed by three times in wash buffer B containing 0.1% w/v nonidet P-40 and 0.5 M NaCl in PBS.

The proteins were than eluted from the beads using PBS containing 50 mM DTT and 62.5 mM Tris HCl. This fraction was designated as cell surface polypeptide fraction.

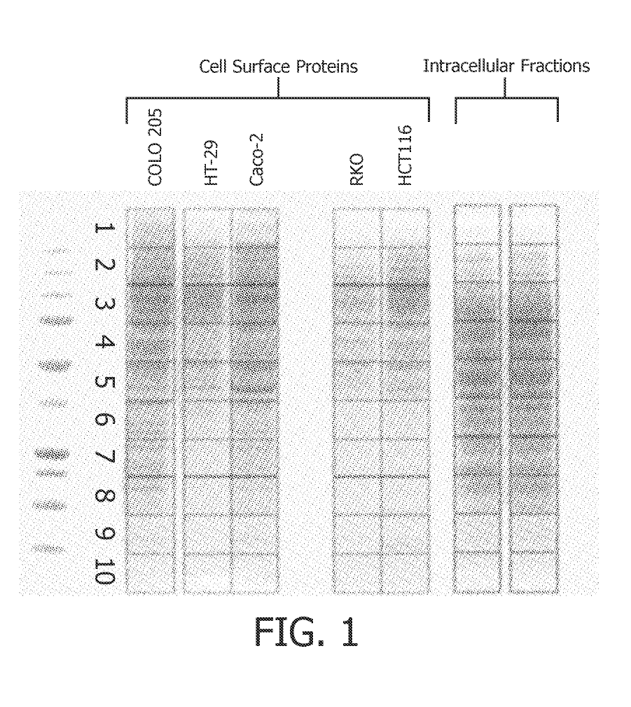

Subsequently, the purified fractions were analysed by SDS-PAGE. An example of biotin-labelled cell surface and intracellular fractions is shown in FIG. 1.

The streptavidine-purified biotin-labelled proteins of the cell surface fraction were then identified by mass spectrometry.

After electrophoresis the gels were fixed in 50% ethanol containing 3% phosphoric acid and stained with Coomassie R-250. After staining the gels were washed in MilliQ water and stored at 4° C. until processing for in-gel digestion.

The gel lanes corresponding to the cell surface proteins of the five different cell lines and the mixtures of intracellular fractions were cut in 10 bands. Each band was processed for in-gel digestion. Briefly, bands were washed dehydrated three times in 50 mM ABC (ammonium bicarbonate pH 7.9)/50 mM ABC+50% ACN (acetonitrile).

Subsequently, cysteine bonds were reduced with 10 mM dithiotreitol for 1 h at 56° C. and alkylated with 50 mM iodoacetamide for 45 min at RT in the dark. After two subsequent wash/dehydration cycles the bands were dried 10 min in a vacuum centrifuge and incubated overnight with 0.06 μg/μl trypsin at 25° C. Peptides were extracted once in 1% formic acid and

Subsequently two times in 50% ACN in 5% formic acid. The volume was reduced to 50 μl in a vacuum centrifuge prior to LC-MS/MS analysis.

Peptides were separated by an Ultimate 3000 nanoLC system (Dionex LC-Packings, Amsterdam, The Netherlands) equipped with a 20 cm×75 μm ID fused silica column custom packed with 3 μm 100 Å ReproSil Pur C18 aqua (Dr Maisch GMBH, Ammerbuch-Entringen, Germany). After injection, peptides were trapped at 30 μl/min on a 5 mm×300 μm ID Pepmap C18 cartridge (Dionex LC-Packings, Amsterdam, The Netherlands) at 2% buffer B (buffer A: 0.05% formic acid in MQ; buffer B: 80% ACN+0.05% formic acid in MQ) and separated at 300 nl/min in a 10-40% buffer B gradient in 60 min.

Eluting peptides were ionized at 1.7 kV in a Nanomate Triversa Chip-based nanospray source using a Triversa LC coupler (Advion, Ithaca, N.J.). Intact peptide mass spectra and fragmentation spectra were acquired on a LTQ-FT hybrid mass spectrometer (Thermo Fisher, Bremen, Germany). Intact masses were measured at resolution 50.000 in the ICR cell using a target value of 1×10⁶ charges. In parallel, following an FT pre-scan, the top 5 peptide signals (charge-states 2+ and higher) were submitted to MS/MS in the linear ion trap (3 amu isolation width, 30 ms activation, 35% normalized activation energy, Q value of 0.25 and a threshold of 5000 counts). Dynamic exclusion was applied with a repeat count of 1 and an exclusion time of 30 s.

MS/MS spectra were searched against the human IPI database 3.31(67511 entries) using Sequest (version 27, rev 12), which is part of the BioWorks 3.3 data analysis package (Thermo Fisher, San Jose, Calif.). MS/MS spectra were searched with a maximum allowed deviation of 10 ppm for the precursor mass and 1 amu for fragment masses. Methionine oxidation and cysteine carboxamidomethylation were allowed as variable modifications. Two missed cleavages were allowed and the minimum number of tryptic termini was 1. After database searching the DTA and OUT files were imported into Scaffold 1.07 (Proteome software, Portland, Oreg.). Scaffold was used to organize the gelband data and to validate peptide identifications using the Peptide Prophet algorithm [14], only identifications with a probabilityN95% were retained. Subsequently, the Protein

Prophet algorithm [15] was applied and protein identifications with a probability of N99% with 2 peptides or more in at least one of the samples were retained. Proteins that contained similar peptides and could not be differentiated based on MS/MS analysis alone were grouped. For each protein identified, the number of unique peptides was exported to Excel. (Piersma et al. (2009) J. Proteomics 72(1): 91-109)

This analysis revealed approximately 2986 proteins.

In parallel, an analysis of genes being implicated in the transgression of adenoma to colorectal adenocarcinoma was performed. This analysis revealed approximately 2478 genes the expression of which is either up- or down-regulated. This approach is described in detail in Carvalho et al. in Gut (2009), 58(1):79-89.

Based on this analysis, only those proteins of the purified cell surface fraction for which the gene analysis data revealed an over-expression were selected. This led to identification of approximately 1305 proteins. Of these 355 genes were over expressed with a significant p-value (>0.05) according to the Wilcox rank test (see Carvalho et al Gut (2009), 58(1):79-89).

In a next step, proteins were selected which comprised at least one transmembrane (TM) domain.

To this end, the software program Phobias developed by the Stockholm Bioinformatics Center was used. (Käll et al. (2004) Journal of Molecular Biology, 338(5):1027-1036). This program indicates whether a protein contains a transmembrane region and/or a signal peptide. Proteins containing either one of these or both were selected.

To reduce the number of false positives we determined which proteins were enriched in the cell surface fractions when compared to the non-biotinylated fractions. A log2 ratio measured from spectral counts (RSC) analysis was carried out. This analysis relied on a label-free quantification approach based on spectral counts. Such an approach is well suited to calculate relative protein abundance between samples and was done according to the method of Old et al. (Old et al (2005) Molecular & Cellular Proteomics 4(10): 1487-1502).

Using this analysis, only proteins with a positive RSC value were selected.

Finally, proteins were selected which were found in at least 70% of the CRC cell lines tested.

This revealed a set of 31 proteins having SEQ ID Nos.: 1 to 32.

A further set of 11 proteins was identified for which the RSC value increased at least by a factor of 2.5. These proteins have SEQ ID Nos. 1 to 10 and SEQ ID No.: 32.

Of these, histological analysis was performed for PRNP (SEQ ID No.: 6) and BCAM (SEQ ID No.: 5) on tumor material obtained from CRC patients.

A 4 mm section of formalin fixed paraffin embedded (FFPE) tissue was used for immunohistochemistry. After deparaffination in xylene, and rehydration through graded alcohol to water, endogenous peroxidase was blocked with hydrogen peroxide (0.3% H2O2/methanol) for 30 min. Antigen retrieval was done by autoclaving in 1.5 mM HCL for PRNP and TRIS/EDTA ph 9 buffer for BCAM. Primary anti-prion mouse clone 8H4 antibody from sigma-aldrich (St. Louis, Mo., USA) was incubated overnight at a dilution of 1:100 and mouse anti human CD239 from abD serotech (Oxford, UK) was also incubated overnight at a dilution of 1:100. Following extensive washing the slides were incubated for 30 min with poly-HRP-goat anti-mouse/rabbit/rat IgG (Powervision, Immunologic, Duiven). Then all sections were washed and incubated with DAB Substrate-Chromogen (Dako, Glostrup, Denmark). Counterstaining was done with Mayer's haematoxylin. Incubation without primary antibody was used as negative control.

The results can be seen in FIG. 2 for PRNP and FIG. 3 for BCAM.

1. Diagnostic marker comprising at least one polypeptide of the group consisting of:
A polypeptide of SEQ ID No.: 1;
A polypeptide of SEQ ID No.: 2;
A polypeptide of SEQ ID No.: 3;
A polypeptide of SEQ ID No.: 4;
A polypeptide of SEQ ID No.: 5;
A polypeptide of SEQ ID No.: 6;
A polypeptide of SEQ ID No.: 7;
A polypeptide of SEQ ID No.: 8;
A polypeptide of SEQ ID No.: 9;
A polypeptide of SEQ ID No.: 10;
A polypeptide of SEQ ID No.: 11;
A polypeptide of SEQ ID No.: 12;
A polypeptide of SEQ ID No.: 13;
A polypeptide of SEQ ID No.: 14;
A polypeptide of SEQ ID No.: 15;
A polypeptide of SEQ ID No.: 16;
A polypeptide of SEQ ID No.: 17;
A polypeptide of SEQ ID No.: 18;
A polypeptide of SEQ ID No.: 19;
A polypeptide of SEQ ID No.: 20;
A polypeptide of SEQ ID No.: 21;
A polypeptide of SEQ ID No.: 22;
A polypeptide of SEQ ID No.: 23;
A polypeptide of SEQ ID No.: 24;
A polypeptide of SEQ ID No.: 25;
A polypeptide of SEQ ID No.: 26;
A polypeptide of SEQ ID No.: 27;
A polypeptide of SEQ ID No.: 28;
A polypeptide of SEQ ID No.: 29;
A polypeptide of SEQ ID No.: 30;
A polypeptide of SEQ ID No.: 31; and/or
A polypeptide of SEQ ID No.: 32.

2. Diagnostic marker of claim 1 comprising at least the polypeptides:
A polypeptide of SEQ ID No.: 1;
A polypeptide of SEQ ID No.: 2; and/or
A polypeptide of SEQ ID No.: 3.

3. Use of at least one polypeptide selected from the group consisting of:
A polypeptide of SEQ ID No.: 1;
A polypeptide of SEQ ID No.: 2;
A polypeptide of SEQ ID No.: 3;
A polypeptide of SEQ ID No.: 4;
A polypeptide of SEQ ID No.: 5;
A polypeptide of SEQ ID No.: 6;
A polypeptide of SEQ ID No.: 7;
A polypeptide of SEQ ID No.: 8;
A polypeptide of SEQ ID No.: 9;
A polypeptide of SEQ ID No.: 10;
A polypeptide of SEQ ID No.: 11;
A polypeptide of SEQ ID No.: 12;
A polypeptide of SEQ ID No.: 13;
A polypeptide of SEQ ID No.: 14;
A polypeptide of SEQ ID No.: 15;
A polypeptide of SEQ ID No.: 16;
A polypeptide of SEQ ID No.: 17;
A polypeptide of SEQ ID No.: 18;
A polypeptide of SEQ ID No.: 19;
A polypeptide of SEQ ID No.: 20;
A polypeptide of SEQ ID No.: 21;
A polypeptide of SEQ ID No.: 22;
A polypeptide of SEQ ID No.: 23;
A polypeptide of SEQ ID No.: 24;
A polypeptide of SEQ ID No.: 25;
A polypeptide of SEQ ID No.: 26;
A polypeptide of SEQ ID No.: 27;
A polypeptide of SEQ ID No.: 28;
A polypeptide of SEQ ID No.: 29;
A polypeptide of SEQ ID No.: 30;
A polypeptide of SEQ ID No.: 31; and/or
A polypeptide of SEQ ID No.: 32.
as a diagnostic marker.

4. Use of claim 3, wherein at least the following polypeptides are used:
A polypeptide of SEQ ID No.: 1;
A polypeptide of SEQ ID No.: 2; and
A polypeptide of SEQ ID No.: 3.

5. A contrast agent, optionally for use in MRI and/or MPI comprising at least one compound being capable of interacting with a polypeptide selected from the group consisting of:
A polypeptide of SEQ ID No.: 1;
A polypeptide of SEQ ID No.: 2;
A polypeptide of SEQ ID No.: 3;
A polypeptide of SEQ ID No.: 4;
A polypeptide of SEQ ID No.: 5;
A polypeptide of SEQ ID No.: 6;
A polypeptide of SEQ ID No.: 7;
A polypeptide of SEQ ID No.: 8;
A polypeptide of SEQ ID No.: 9;
A polypeptide of SEQ ID No.: 10;
A polypeptide of SEQ ID No.: 11;
A polypeptide of SEQ ID No.: 12;
A polypeptide of SEQ ID No.: 13;
A polypeptide of SEQ ID No.: 14;
A polypeptide of SEQ ID No.: 15;
A polypeptide of SEQ ID No.: 16;
A polypeptide of SEQ ID No.: 17;
A polypeptide of SEQ ID No.: 18;
A polypeptide of SEQ ID No.: 19;
A polypeptide of SEQ ID No.: 20;
A polypeptide of SEQ ID No.: 21;
A polypeptide of SEQ ID No.: 22;
A polypeptide of SEQ ID No.: 23;
A polypeptide of SEQ ID No.: 24;
A polypeptide of SEQ ID No.: 25;
A polypeptide of SEQ ID No.: 26;
A polypeptide of SEQ ID No.: 27;
A polypeptide of SEQ ID No.: 28;
A polypeptide of SEQ ID No.: 29;
A polypeptide of SEQ ID No.: 30;
A polypeptide of SEQ ID No.: 31; and/or
A polypeptide of SEQ ID No.: 32.

6. Contrast agent of claim 5, wherein the contrast agent comprises a compound/compounds which interacts/interact at least with:
A polypeptide of SEQ ID No.: 1;
A polypeptide of SEQ ID No.: 2; and
A polypeptide of SEQ ID No.: 3.

7. Contrast agent of claim 5, wherein the compound/compounds is/are selected from antibodies which specifically interact with at least one polypeptide of SEQ ID No. 1 to 32.

8. Contrast agent of claim 5, wherein the compound/compounds is/are coupled to a detectable marker molecule.

9. Use of at least one antibody capable of interacting with a polypeptide selected from the group consisting of:
A polypeptide of SEQ ID No.: 1;
A polypeptide of SEQ ID No.: 2;
A polypeptide of SEQ ID No.: 3;
A polypeptide of SEQ ID No.: 4;
A polypeptide of SEQ ID No.: 5;
A polypeptide of SEQ ID No.: 6;
A polypeptide of SEQ ID No.: 7;
A polypeptide of SEQ ID No.: 8;
A polypeptide of SEQ ID No.: 9;
A polypeptide of SEQ ID No.: 10;
A polypeptide of SEQ ID No.: 11;
A polypeptide of SEQ ID No.: 12;
A polypeptide of SEQ ID No.: 13;
A polypeptide of SEQ ID No.: 14;
A polypeptide of SEQ ID No.: 15;
A polypeptide of SEQ ID No.: 16;
A polypeptide of SEQ ID No.: 17;
A polypeptide of SEQ ID No.: 18;
A polypeptide of SEQ ID No.: 19;
A polypeptide of SEQ ID No.: 20;
A polypeptide of SEQ ID No.: 21;
A polypeptide of SEQ ID No.: 22;
A polypeptide of SEQ ID No.: 23;
A polypeptide of SEQ ID No.: 24;
A polypeptide of SEQ ID No.: 25;
A polypeptide of SEQ ID No.: 26;
A polypeptide of SEQ ID No.: 27;
A polypeptide of SEQ ID No.: 28;
A polypeptide of SEQ ID No.: 29;
A polypeptide of SEQ ID No.: 30;
A polypeptide of SEQ ID No.: 31; and/or
A polypeptide of SEQ ID No.: 32
as a contrast agent, optionally for use MRI and/or MPI.

10. Use of claim 9, wherein the antibody/antibodies is/are selected from commercially available antibodies which interact with the said polypeptides.

11. Use of claim 9, wherein the antibody/antibodies is/are coupled to a detectable marker molecule.

12. Method of diagnosing colorectal cancer (CRC) comprising at least the following steps:
a) Obtaining at least one sample from at least one human or animal individual suspected to suffer from ongoing or imminent CRC development;
b) Testing in said at least one sample for expression of at least one polypeptide selected from the group consisting of:
A polypeptide of SEQ ID No.: 1;
A polypeptide of SEQ ID No.: 2;
A polypeptide of SEQ ID No.: 3;
A polypeptide of SEQ ID No.: 4;
A polypeptide of SEQ ID No.: 5;
A polypeptide of SEQ ID No.: 6;
A polypeptide of SEQ ID No.: 7;
A polypeptide of SEQ ID No.: 8;
A polypeptide of SEQ ID No.: 9;
A polypeptide of SEQ ID No.: 10;
A polypeptide of SEQ ID No.: 11;
A polypeptide of SEQ ID No.: 12;
A polypeptide of SEQ ID No.: 13;
A polypeptide of SEQ ID No.: 14;
A polypeptide of SEQ ID No.: 15;
A polypeptide of SEQ ID No.: 16;
A polypeptide of SEQ ID No.: 17;
A polypeptide of SEQ ID No.: 18;
A polypeptide of SEQ ID No.: 19;
A polypeptide of SEQ ID No.: 20;
A polypeptide of SEQ ID No.: 21;
A polypeptide of SEQ ID No.: 22;
A polypeptide of SEQ ID No.: 23;
A polypeptide of SEQ ID No.: 24;
A polypeptide of SEQ ID No.: 25;
A polypeptide of SEQ ID No.: 26;
A polypeptide of SEQ ID No.: 27;
A polypeptide of SEQ ID No.: 28;
A polypeptide of SEQ ID No.: 29;
A polypeptide of SEQ ID No.: 30;
A polypeptide of SEQ ID No.: 31; and/or
A polypeptide of SEQ ID No.: 32;
c) Testing in at least one control sample obtained from at least one human or animal individual not suffering from ongoing or imminent CRC development for expression of at least one polypeptide selected from the group consisting of:
A polypeptide of SEQ ID No.: 1;
A polypeptide of SEQ ID No.: 2;
A polypeptide of SEQ ID No.: 3;
A polypeptide of SEQ ID No.: 4;
A polypeptide of SEQ ID No.: 5;
A polypeptide of SEQ ID No.: 6;
A polypeptide of SEQ ID No.: 7;
A polypeptide of SEQ ID No.: 8;
A polypeptide of SEQ ID No.: 9;
A polypeptide of SEQ ID No.: 10;
A polypeptide of SEQ ID No.: 11;
A polypeptide of SEQ ID No.: 12;
A polypeptide of SEQ ID No.: 13;
A polypeptide of SEQ ID No.: 14;
A polypeptide of SEQ ID No.: 15;
A polypeptide of SEQ ID No.: 16;
A polypeptide of SEQ ID No.: 17;
A polypeptide of SEQ ID No.: 18;
A polypeptide of SEQ ID No.: 19;
A polypeptide of SEQ ID No.: 20;
A polypeptide of SEQ ID No.: 21;
A polypeptide of SEQ ID No.: 22;
A polypeptide of SEQ ID No.: 23;
A polypeptide of SEQ ID No.: 24;
A polypeptide of SEQ ID No.: 25;
A polypeptide of SEQ ID No.: 26;
A polypeptide of SEQ ID No.: 27;
A polypeptide of SEQ ID No.: 28;
A polypeptide of SEQ ID No.: 29;
A polypeptide of SEQ ID No.: 30;
A polypeptide of SEQ ID No.: 31; and/or
A polypeptide of SEQ ID No.: 32;
d) Determining difference in expression of steps b) and d);
e) Deciding on the presence or imminence of CRC development based on the results obtained in step d).

13. Method of diagnosing CRC comprising at least the following steps:
a) Testing in at least one human or animal individual suspected to suffer from ongoing or imminent CRC development for expression of at least one polypeptide selected from the group consisting of:
A polypeptide of SEQ ID No.: 1;
A polypeptide of SEQ ID No.: 2;
A polypeptide of SEQ ID No.: 3;
A polypeptide of SEQ ID No.: 4;
A polypeptide of SEQ ID No.: 5;
A polypeptide of SEQ ID No.: 6;
A polypeptide of SEQ ID No.: 7;
A polypeptide of SEQ ID No.: 8;
A polypeptide of SEQ ID No.: 9;
A polypeptide of SEQ ID No.: 10;
A polypeptide of SEQ ID No.: 11;
A polypeptide of SEQ ID No.: 12;
A polypeptide of SEQ ID No.: 13;
A polypeptide of SEQ ID No.: 14;
A polypeptide of SEQ ID No.: 15;
A polypeptide of SEQ ID No.: 16;
A polypeptide of SEQ ID No.: 17;
A polypeptide of SEQ ID No.: 18;
A polypeptide of SEQ ID No.: 19;
A polypeptide of SEQ ID No.: 20;
A polypeptide of SEQ ID No.: 21;
A polypeptide of SEQ ID No.: 22;
A polypeptide of SEQ ID No.: 23;
A polypeptide of SEQ ID No.: 24;
A polypeptide of SEQ ID No.: 25;
A polypeptide of SEQ ID No.: 26;
A polypeptide of SEQ ID No.: 27;
A polypeptide of SEQ ID No.: 28;
A polypeptide of SEQ ID No.: 29;
A polypeptide of SEQ ID No.: 30;
A polypeptide of SEQ ID No.: 31; and/or
A polypeptide of SEQ ID No.: 32;
b) Comparing expression as determined in step a) with expression of at least one polypeptide selected from the group consisting of:
A polypeptide of SEQ ID No.: 1;
A polypeptide of SEQ ID No.: 2;
A polypeptide of SEQ ID No.: 3;
A polypeptide of SEQ ID No.: 4;
A polypeptide of SEQ ID No.: 5;
A polypeptide of SEQ ID No.: 6;
A polypeptide of SEQ ID No.: 7;
A polypeptide of SEQ ID No.: 8;
A polypeptide of SEQ ID No.: 9;
A polypeptide of SEQ ID No.: 10;
A polypeptide of SEQ ID No.: 11;
A polypeptide of SEQ ID No.: 12;
A polypeptide of SEQ ID No.: 13;
A polypeptide of SEQ ID No.: 14;
A polypeptide of SEQ ID No.: 15;
A polypeptide of SEQ ID No.: 16;
A polypeptide of SEQ ID No.: 17;
A polypeptide of SEQ ID No.: 18;
A polypeptide of SEQ ID No.: 19;
A polypeptide of SEQ ID No.: 20;
A polypeptide of SEQ ID No.: 21;
A polypeptide of SEQ ID No.: 22;
A polypeptide of SEQ ID No.: 23;
A polypeptide of SEQ ID No.: 24;
A polypeptide of SEQ ID No.: 25;
A polypeptide of SEQ ID No.: 26;
A polypeptide of SEQ ID No.: 27;
A polypeptide of SEQ ID No.: 28;
A polypeptide of SEQ ID No.: 29;
A polypeptide of SEQ ID No.: 30;
A polypeptide of SEQ ID No.: 31; and/or
A polypeptide of SEQ ID No.: 32;
as determined for human or animal individuals not suffering from ongoing or imminent colorectal cancer development
c) Deciding on the presence or imminence of CRCdevelopment based on the results obtained in step b).

14. Method of data acquisition comprising at least the following steps:
a) Testing in at least one human or animal individual suspected to suffer from ongoing or imminent CRC development for expression of at least one polypeptide selected from the group consisting of:
A polypeptide of SEQ ID No.: 1;
A polypeptide of SEQ ID No.: 2;
A polypeptide of SEQ ID No.: 3;
A polypeptide of SEQ ID No.: 4;
A polypeptide of SEQ ID No.: 5;
A polypeptide of SEQ ID No.: 6;
A polypeptide of SEQ ID No.: 7;
A polypeptide of SEQ ID No.: 8;
A polypeptide of SEQ ID No.: 9;
A polypeptide of SEQ ID No.: 10;
A polypeptide of SEQ ID No.: 11;
A polypeptide of SEQ ID No.: 12;
A polypeptide of SEQ ID No.: 13;
A polypeptide of SEQ ID No.: 14;
A polypeptide of SEQ ID No.: 15;
A polypeptide of SEQ ID No.: 16;
A polypeptide of SEQ ID No.: 17;
A polypeptide of SEQ ID No.: 18;
A polypeptide of SEQ ID No.: 19;
A polypeptide of SEQ ID No.: 20;
A polypeptide of SEQ ID No.: 21;
A polypeptide of SEQ ID No.: 22;
A polypeptide of SEQ ID No.: 23;
A polypeptide of SEQ ID No.: 24;
A polypeptide of SEQ ID No.: 25;
A polypeptide of SEQ ID No.: 26;
A polypeptide of SEQ ID No.: 27;
A polypeptide of SEQ ID No.: 28;
A polypeptide of SEQ ID No.: 29;
A polypeptide of SEQ ID No.: 30;
A polypeptide of SEQ ID No.: 31; and/or
A polypeptide of SEQ ID No.: 32;
b) Comparing expression as determined in step a) with expression of at least one polypeptide selected from the group consisting of:
A polypeptide of SEQ ID No.: 1;
A polypeptide of SEQ ID No.: 2;
A polypeptide of SEQ ID No.: 3;
A polypeptide of SEQ ID No.: 4;
A polypeptide of SEQ ID No.: 5;
A polypeptide of SEQ ID No.: 6;
A polypeptide of SEQ ID No.: 7;
A polypeptide of SEQ ID No.: 8;
A polypeptide of SEQ ID No.: 9;
A polypeptide of SEQ ID No.: 10;
A polypeptide of SEQ ID No.: 11;
A polypeptide of SEQ ID No.: 12;
A polypeptide of SEQ ID No.: 13;
A polypeptide of SEQ ID No.: 14;
A polypeptide of SEQ ID No.: 15;
A polypeptide of SEQ ID No.: 16;
A polypeptide of SEQ ID No.: 17;
A polypeptide of SEQ ID No.: 18;
A polypeptide of SEQ ID No.: 19;
A polypeptide of SEQ ID No.: 20;
A polypeptide of SEQ ID No.: 21;
A polypeptide of SEQ ID No.: 22;
A polypeptide of SEQ ID No.: 23;
A polypeptide of SEQ ID No.: 24;
A polypeptide of SEQ ID No.: 25;
A polypeptide of SEQ ID No.: 26;
A polypeptide of SEQ ID No.: 27;
A polypeptide of SEQ ID No.: 28;
A polypeptide of SEQ ID No.: 29;
A polypeptide of SEQ ID No.: 30;
A polypeptide of SEQ ID No.: 31; and/or
A polypeptide of SEQ ID No.: 32;
as determined for human or animal individuals not suffering from ongoing or imminent CRC development

15. Use of diagnostic markers of claim 1 for differentiating progressive (high-risk) CRC (adenocarcinomas) from non-progressive (low-risk) colorectal adenomas.


SEQUENCE LISTING (32 sequences)

SEQ ID NO: 1 - protein, 1896 aa, Homo sapiens
MPLPPRSLQVLLLLLLLLLLLPGMWAEAGLPRAGGGSQPPFRTFSASDWGLTHLVVHEQTGEVYVGAVNRIYKLSGNLTLLRAHVTGPVEDNEKCYPPPSVQSCPHGLGSTDNVNKLLLLDYAANRLLACGSASQGICQFLRLDDLFKLGEPHHRKEHYLSSVQEAGSMAGVLIAGPPGQGQAKLFVGTPIDGKSEYFPTLSSRRLMANEEDADMFGFVYQDEFVSSQLKIPSDTLSKFPAFDIYYVYSFRSEQFVYYLTLQLDTQLTSPDAAGEHFFTSKIVRLCVDDPKFYSYVEFPIGCEQAGVEYRLVQDAYLSRPGRALAHQLGLAEDEDVLFTVFAQGQKNRVKPPKESALCLFTLRAIKEKIKERIQSCYRGEGKLSLPWLLNKELGCINSPLQIDDDFCGQDFNQPLGGTVTIEGTPLFVDKDDGLTAVAAYDYRGRTVVFAGTRSGRIRKILVDLSNPGGRPALAYESVVAQEGSPILRDLVLSPNHQYLYAMTEKQVTRVPVESCVQYTSCELCLGSRDPHCGWCVLHSICSRRDACERADEPQRFAADLLQCVQLTVQPRNVSVTMSQVPLVLQAWNVPDLSAGVNCSFEDFTESESVLEDGRIHCRSPSAREVAPITRGQGDQRVVKLYLKSKETGKKFASVDFVFYNCSVHQSCLSCVNGSFPCHWCKYRHVCTHNVADCAFLEGRVNVSEDCPQILPSTQIYVPVGVVKPITLAARNLPQPQSGQRGYECLFHIPGSPARVTALRFNSSSLQCQNSSYSYEGNDVSDLPVNLSVVWNGNFVIDNPQNIQAHLYKCPALRESCGLCLKADPRFECGWCVAERRCSLRHHCAADTPASWMHARHGSSRCTDPKILKLSPETGPRQGGTRLTITGENLGLRFEDVRLGVRVGKVLCSPVESEYISAEQIVCEIGDASSVRAHDALVEVCVRDCSPHYRALSPKRFTFVTPTFYRVSPSRGPLSGGTWIGIEGSHLNAGSDVAVSVGGRPCSFSWRNSREIRCLTPPGQSPGSAPIIININRAQLTNPEVKYNYTEDPTILRIDPEWSINSGGTLLTVTGTNLATVREPRIRAKYGGIERENGCLVYNDTTMVCRAPSVANPVRSPPELGERPDELGFVMDNVRSLLVLNSTSFLYYPDPVLEPLSPTGLLELKPSSPLILKGRNLLPPAPGNSRLNYTVLIGSTPCTLTVSETQLLCEAPNLTGQHKVTVRAGGFEFSPGTLQVYSDSLLTLPAIVGIGGGGGLLLLVIVAVLIAYKRKSRDADRTLKRLQLQMDNLESRVALECKEAFAELQTDIHELTNDLDGAGIPFLDYRTYAMRVLFPGIEDHPVLKEMEVQANVEKSLTLFGQLLTKKHFLLTFIRTLEAQRSFSMRDRGNVASLIMTALQGEMEYATGVLKQLLSDLIEKNLESKNHPKLLLRRTESVAEKMLTNWFTFLLYKFLKECAGEPLFMLYCAIKQQMEKGPIDAITGEARYSLSEDKLIRQQIDYKTLTLNCVNPENENAPEVPVKGLDCDTVTQAKEKLLDAAYKGVPYSQRPKAADMDLEWRQGRMARIILQDEDVTTKIDNDWKRLNTLAHYQVTDGSSVALVPKQTSAYNISNSSTFTKSLSRYESMLRTASSPDSLRSRTPMITPDLESGTKLWHLVKNHDHLDQREGDRGSKMVSEIYLTRLLATKGTLQKFVDDLFETIFSTAHRGSALPLAIKYMFDFLDEQADKHQIHDADVRHTWKSNCLPLRFWVNVIKNPQFVFDIHKNSITDACLSVVAQTFMDSCSTSEHKLGKDSPSNKLLYAKDIPNYKSWVERYYADIAKMPAISDQDMSAYLAEQSRLHLSQFNSMSALHEIYSYITKYKDEILAALEKDEQARRQRLRSKLEQVVDTMALSS

SEQ ID NO: 2 - protein, 541 aa, Homo sapiens
MVADPPRDSKGLAAAEPTANGGLALASIEDQGAAAGGYCGSRDQVRRCLRANLLVLLTVVAVVAGVALGLGVSGAGGALALGPERLSAFVFPGELLLRLLRMIILPLVVCSLIGGAASLDPGALGRLGAWALLFFLVTTLLASALGVGLALALQPGAASAAINASVGAAGSAENAPSKEVLDSFLDLARNIFPSNLVSAAFRSYSTTYEERNITGTRVKVPVGQEVEGMNILGLVVFAIVFGVALRKLGPEGELLIRFFNSFNEATMVLVSWIMWYAPVGIMFLVAGKIVEMEDVGLLFARLGKYILCCLLGHAIHGLLVLPLIYFLFTRKNPYRFLWGIVTPLATAFGTSSSSATLPLMMKCVEENNGVAKHISRFILPIGATVNMDGAALFQCVAAVFIAQLSQQSLDFVKIITILVTATASSVGAAGIPAGGVLTLAIILEAVNLPVDHISLILAVDWLVDRSCTVLNVEGDALGAGLLQNYVDRTESRSTEPELIQVKSELPLDPLPVPTEEGNPLLKHYRGPAGDATVASEKESVM

SEQ ID NO: 3 - protein, 509 aa, Homo sapiens
MGCSAKARWAAGALGVAGLLCAVLGAVMIVMVPSLIKQQVLKNVRIDPSSLSFNMWKEIPIPFYLSVYFFDVMNPSEILKGEKPQVRERGPYVYREFRHKSNITFNNNDTVSFLEYRTFQFQPSKSHGSESDYIVMPNILVLGAAVMMENKPMTLKLIMTLAFTTLGERAFMNRTVGEIMWGYKDPLVNLINKYFPGMFPFKDKFGLFAELNNSDSGLFTVFTGVQNISRIHLVDKWNGLSKVDFWHSDQCNMINGTSGQMWPPFMTPESSLEFYSPEACRSMKLMYKESGVFEGIPTYRFVAPKTLFANGSIYPPNEGFCPCLESGIQNVSTCRFSAPLFLSHPHFLNADPVLAEAVTGLHPNQEAHSLFLDIHPVTGIPMNCSVKLQLSLYMKSVAGIGQTGKIEPVVLPLLWFAESGAMEGETLHTFYTQLVLMPKVMHYAQYVLLALGCVLLLVPVICQIRSQEKCYLFWSSSKKGSKDKEAIQAYSESLMTSAPKGSVLQEAKL

SEQ ID NO: 4 - protein, 532 aa, Homo sapiens
MAPSSPRPALPALLVLLGALFPGPGNAQTSVSPSKVILPRGGSVLVTCSTSCDQPKLLGIETPLPKKELLLPGNNRKVYELSNVQEDSQPMCYSNCPDGQSTAKTFLTVYWTPERVELAPLPSWQPVGKNLTLRCQVEGGAPRANLTVVLLRGEKELKREPAVGEPAEVTTTVLVRRDHHGANFSCRTELDLRPQGLELFENTSAPYQLQTFVLPATPPQLVSPRVLEVDTQGTVVCSLDGLFPVSEAQVHLALGDQRLNPTVTYGNDSFSAKASVSVTAEDEGTQRLTCAVILGNQSQETLQTVTIYSFPAPNVILTKPEVSEGTEVTVKCEAHPRAKVTLNGVPAQPLGPRAQLLLKATPEDNGRSFSCSATLEVAGQLIHKNQTRELRVLYGPRLDERDCPGNWTWPENSQQTPMCQAWGNPLPELKCLKDGTFPLPIGESVTVTRDLEGTYLCRARSTQGEVTRKVTVNVLSPRYEIVIITVVAAAVIMGTAGLSTYLYNRQRKIKKYRLQQAQKGTPMKPNTQATPP

SEQ ID NO: 5 - protein, 628 aa, Homo sapiens
MEPPDAPAQARGAPRLLLLAVLLAAHPDAQAEVRLSVPPLVEVMRGKSVILDCTPTGTHDHYMLEWFLTDRSGARPRLASAEMQGSELQVTMHDTRGRSPPYQLDSQGRLVLAEAQVGDERDYVCVVRAGAAGTAEATARLNVFAKPEATEVSPNKGTLSVMEDSAQEIATCNSRNGNPAPKITWYRNGQRLEVPVEMNPEGYMTSRTVREASGLLSLTSTLYLRLRKDDRDASFHCAAHYSLPEGRHGRLDSPTFHLTLHYPTEHVQFWVGSPSTPAGWVREGDTVQLLCRGDGSPSPEYTLFRLQDEQEEVLNVNLEGNLTLEGVTRGQSGTYGCRVEDYDAADDVQLSKTLELRVAYLDPLELSEGKVLSLPLNSSAVVNCSVHGLPTPALRWTKDSTPLGDGPMLSLSSITFDSNGTYVCEASLPTVPVLSRTQNFTLLVQGSPELKTAEIEPKADGSWREGDEVTLICSARGHPDPKLSWSQLGGSPAEPIPGRQGWVSSSLTLKVTSALSRDGISCEASNPHGNKRHVFHFGTVSPQTSQAGVAVMAVAVSVGLLLLVVAVFYCVRRKGGPCCRQRREKGAPPPGEPGLSHSGSEQPEQTGLLMGGASGGARGGSGGFGDEC

SEQ ID NO: 6 - protein, 253 aa, Homo sapiens
MANLGCWMLVLFVATWSDLGLCKKRPKPGGWNTGGSRYPGQGSPGGNRYPPQGGGGWGQPHGGGWGQPHGGGWGQPHGGGWGQPHGGGWGQGGGTHSQWNKPSKPKTNMKHMAGAAAAGAVVGGLGGYMLGSAMSRPIIHFGSDYEDRYYRENMHRYPNQVYYRPMDEYSNQNNFVHDCVNITIKQHTVTTTTKGENFTETDVKMMERVVEQMCITQYERESQAYYQRGSSMVLFSSPPVILLISFLIFLIVG

SEQ ID NO: 7 - protein, 895 aa, Homo sapiens
MRMSVGLSLLLPLSGRTFLLLLSVVMAQSHWPSEPSEAVRDWENQLEASMHSVLSDLHEAVPTVVGIPDGTAVVGRSFRVTIPTDLIASSGDIIKVSAAGKEALPSWLHWDSQSHTLEGLPLDTDKGVHYISVSATRLGANGSHIPQTSSVFSIEVYPEDHSELQSVRTASPDPGEVVSSACAADEPVTVLTVILDADLTKMTPKQRIDLLHRMRSFSEVELHNMKLVPVVNNRLFDMSAFMAGPGNAKKVVENGALLSWKLGCSLNQNSVPDIHGVEAPAREGAMSAQLGYPVVGWHIANKKPPLPKRVRRQIHATPTPVTAIGPPTTAIQEPPSRIVPTPTSPAIAPPTETMAPPVRDPVPGKPTVTIRTRGAIIQTPTLGPIQPTRVSEAGTTVPGQIRPTMTIPGYVEPTAVATPPTTTTKKPRVSTPKPATPSTDSTTTTTRRPTKKPRTPRPVPRVTTKVSITRLETASPPTRIRTTTSGVPRGGEPNQRPELKNHIDRVDAWVGTYFEVKIPSDTFYDHEDTTTDKLKLTLKLREQQLVGEKSWVQFNSNSQLMYGLPDSSHVGKHEYFMHATDKGGLSAVDAFEIHVHRRPQGDRAPARFKAKFVGDPALVLNDIHKKIALVKKLAFAFGDRNCSTITLQNITRGSIVVEWTNNTLPLEPCPKEQIAGLSRRIAEDDGKPRPAFSNALEPDFKATSITVTGSGSCRHLQFIPVVPPRRVPSEAPPTEVPDRDPEKSSEDDVYLHTVIPAVVVAAILLIAGIIAMICYRKKRKGKLTLEDQATFIKKGVPIIFADELDDSKPPPSSSMPLILQEEKAPLPPPEYPNQSVPETTPLNQDTMGEYTPLRDEDPNAPPYQPPPPFTAPMEGKGSRPKNMTPYRSPPPYVPP

SEQ ID NO: 8 - protein, 926 aa, Homo sapiens
SAREESGAGARPRRRSADSGAAGAGRGGGGEAAGKEEEGESRSRRASMGRLASRPLLLALLSLALCRGRVVRVPTATLVRVVGTELVIPCNVSDYDGPSEQNFDWSFSSLGSSFVELASTWEVGFPAQLYQERLQRGEILLRRTANDAVELHIKNVQPSDQGHYKCSTPSTDATVQGNYEDTVQVKVLADSLHVGPSARPPPSLSLREGEPFELRCTAASASPLHTHLALLWEVHRGPARRSVLALTHEGRFHPGLGYEQRYHSGDVRLDTVGSDAYRLSVSRALSADQGSYRCIVSEWIAEQGNWQEIQEKAVEVATVVIQPSVLRAAVPKNVSVAEGKELDLTCNITTDRADDVRPEVTWSFSRMPDSTLPGSRVLARLDRDSLVHSSPHVALSHVDARSYHLLVRDVSKENSGYYYCHVSLWAPGHNRSWHKVAEAVSSPAGVGVTWLEPDYQVYLNASKVPGFADDPTELACRVVDTKSGEANVRFTVSWYYRMNRRSDNVVTSELLAVMDGDWTLKYGERSKQRAQDGDFIFSKEHTDTFNFRIQRTTEEDRGNYYCVVSAWTKQRNNSWVKSKDVFSKPVNIFWALEDSVLVVKARQPKPFFAAGNTFEMTCKVSSKNIKSPRYSVLIMAEKPVGDLSSPNETKYIISLDQDSVVKLENWTDASRVDGVVLEKVQEDEFRYRMYQTQVSDAGLYRCMVTAWSPVRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 9 - protein, 613 aa, Homo sapiens
MGALRPTLLPPSLPLLLLLMLGMGCWAREVLVPEGPLYRVAGTAVSISCNVTGYEGPAQQNFEWFLYRPEAPDTALGIVSTKDTQFSYAVFKSRVVAGEVQVQRLQGDAVVLKIARLQAQDAGIYECHTPSTDTRYLGSYSGKVELRVLPDVLQVSAAPPGPRGRQAPTSPPRMTVHEGQELALGCLARTSTQKHTHLAVSFGRSVPEAPVGRSTLQEVVGIRSDLAVEAGAPYAERLAAGELRLGKEGTDRYRMVVGGAQAGDAGTYHCTAAEWIQDPDGSWAQIAEKRAVLAHVDVQTLSSQLAVTVGPGERRIGPGEPLELLCNVSGALPPAGRHAAYSVGWEMAPAGAPGPGRLVAQLDTEGVGSLGPGYEGRHIAMEKVASRTYRLRLEAARPGDAGTYRCLAKAYVRGSGTRLREAASARSRPLPVHVREEGVVLEAVAWLAGGTVYRGETASLLCNISVRGGPPGLRLAASWWVERPEDGELSSVPAQLVGGVGQDGVAELGVRPGGGPVSVELVGPRSHRLRLHSLGPEDEGVYHCAPSAWVQHADYSWYQAGSARSGPVTVYPYMHALDTLFVPLLVGTGVALVTGATVLGTITCCFMKRLRKR

SEQ ID NO: 10 - protein, 860 aa, Homo sapiens [sequence omitted]

SEQ ID NO: 11 - protein, 542 aa, Homo sapiens
MTKSNGEEPKMGGRMERFQQGVRKRTLLAKKKVQNITKEDVKSYLFRNAFVLLTVTAVIVGTILGFTLRPYRMSYREVKYFSFPGELLMRMLQMLVLPLIISSLVTGMAALDSKASGKMGMRAVVYYMTTTIIAVVIGIIIVIIIHPGKGTKENMHREGKIVRVTAADAFLDLIRNMFPPNLVEACFKQFKTNYEKRSFKVPIQANETLVGAVINNVSEAMETLTRITEELVPVPGSVNGVNALGLVVFSMCFGFVIGNMKEQGQALREFFDSLNEAIMRLVAVIMWYAPVGILFLIAGKIVEMEDMGVIGGQLAMYTVTVIVGLLIHAVIVLPLLYFLVTRKNPWVFIGGLLQALITALGTSSSSATLPITFKCLEENNGVDKRVTRFVLPVGATINMDGTALYEALAAIFIAQVNNFELNFGQIITISITATAASIGAAGIPQAGLVTMVIVLTSVGLPTDDITLIIAVDWFLDRLRTTTNVLGDSLGAGIVEHLSRHELKNRDVEMGNSVIEENEMKKPYQLIAQDNETEKPIDSETKM

SEQ ID NO: 12 - protein, 698 aa, Homo sapiens
MQAHELFRYFRMPELVDFRQYVRTLPTNTLMGFGAFAALTTFWYATRPKPLKPPCDLSMQSVEVAGSGGARRSALLDSDEPLVYFYDDVTTLYEGFQRGIQVSNNGPCLGSRKPDQPYEWLSYKQVAELSECIGSALIQKGFKTAPDQFIGIFAQNRPEWVIIEQGCFAYSMVIVPLYDTLGNEAITYIVNKAELSLVFVDKPEKAKLLLEGVENKLIPGLKIIVVMDAYGSELVERGQRCGVEVTSMKAMEDLGRANRRKPKPPAPEDLAVICFTSGTTGNPKGAMVTHRNIVSDCSAFVKATENTVNPCPDDTLISFLPLAHMFERVVECVMLCHGAKIGFFQGDIRLLMDDLKVLQPTVFPVVPRLLNRMFDRIFGQANTTLKRWLLDFASKRKEAELRSGIIRNNSLWDRLIFHKVQSSLGGRVRLMVTGAAPVSATVLTFLRAALGCQFYEGYGQTECTAGCCLTMPGDWTAGHVGAPMPCNLIKLVDVEEMNYMAAEGEGEVCVKGPNVFQGYLKDPAKTAEALDKDGWLHTGDIGKWLPNGTLKIIDRKKHIFKLAQGEYIAPEKIENIYMRSEPVAQVFVHGESLQAFLIAIVVPDVETLCSWAQKRGFEGSFEELCRNKDVKKAILEDMVRLGKDSGLKPFEQVKGITLHPELFSIDNGLLTPTMKAKRPELRNYFRSQIDDLYSTIKV

SEQ ID NO: 13 - protein, 552 aa, Homo sapiens
MLDHKDLEAEIHPLKNEERKSQENLGNPSKNEDNVKSAPPQSRLSRCRAAAFFLSLFLCLFVVFVVSFVIPCPDRPASQRMWRIDYSAAVIYDFLAVDDINGDRIQDVLFLYKNTNSSNNFSRSCVDEGFSSPCTFAAAVSGANGSTLWERPVAQDVALVECAVPQPRGSEAPSACILVGRPSSFIAVNLFTGETLWNHSSSFSGNASILSPLLQVPDVDGDGAPDLLVLTQEREEVSGHLYSGSTGHQIGLRGSLGVDGESGFLLHVTRTGAHYILFPCASSLCGCSVKGLYEKVTGSGGPFKSDPHWESMLNATTRRMLSHSSGAVRYLMHVPGNAGADVLLVGSEAFVLLDGQELTPRWTPKAAHVLRKPIFGRYKPDTLAVAVENGTGTDRQILFLDLGTGAVLCSLALPSLPGGPLSASLPTADHRSAFFFWGLHELGSTSETETGEARHSLYMFHPTLPRVLLELANVSTHIVAFDAVLFEPSRHAAYILLTGPADSEAPGLVSVIKHKVRDLVPSSRVVRLGEGGPDSDQAIRDRFSRLRYQSEA

SEQ ID NO: 14 - protein, 963 aa, Homo sapiens
MGLPEPGPLRLLALLLLLLLLLLLQLQHLAAAAADPLLGGQGPAKDCEKDQFQCRNERCIPSVWRCDEDDDCLDHSDEDDCPKKTCADSDFTCDNGHCIHERWKCDGEEECPDGSDESEATCTKQVCPAEKLSCGPTSHKCVPASWRCDGEKDCEGGADEAGCATLCAPHEFQCGNRSCLAAVFVCDGDDDCGDGSDERGCADPACGPREFRCGGDGGGACIPERWVCDRQFDCEDRSDEAAELCGRPGPGATSAPAACATASQFACRSGECVHLGWRCDGDRDCKDKSDEADCPLGTCRGDEFQCGDGTCVLAIKHCNQEQDCPDGSDEAGCLQGLNECLHNNGGCSHICTDLKIGFECTCPAGFQLLDQKTCGDIDECKDPDACSQICVNYKGYFKCECYPGYEMDLLTKNCKAAAGKSPSLIFTNRHEVRRIDLVKRNYSRLIPMLKNVVALDVEVATNRIYWCDLSYRKIYSAYMDKASDPKEQEVLIDEQLHSPEGLAVDWVHKHIYWTDSGNKTISVATVDGGRRRTLFSRNLSEPRAIAVDPLRGFMYWSDWGDQAKIEKSGLNGVDRQTLVSDNIEWPNGITLDLLSQRLYWVDSKLHQLSSIDFSGGNRKTLISSTDFLSHPFGIAVFEDKVFWTDLENEAIFSANRLNGLEISILAENLNNPHDIVIFHELKQPRAPDACELSVQPNGGCEYLCLPAPQISSHSPKYTCACPDTMWLGPDMKRCYRAPQSTSTTTLASTMTRTVPATTRAPGTTVHRSTYQNHSTETPSLTAAVPSSVSVPRAPSISPSTLSPATSNHSQHYANEDSKMGSTVTAAVIGIIVPIVVIALLCMSGYLIWRNWKRKNTKSMNFDNPVYRKTTEEEDEDELHIGRTAQIGHVYPAAISSFDRPLWAEPCLGETREPEDPAPALKELFVLPGEPRSQLHQLPKNPLSELPVVKSKRVALSLEDDGLP

SEQ ID NO: 15 - protein, 150 aa, Homo sapiens
MSGSMATAEASGSDGKGQEVETSVTYYRLEEVAKRNSLKELWLVIHGRVYDVTRFLNEHPGGEEVLLEQAGVDASESFEDVGHSSDAREMLKQYYIGDIHPSDLKPESGSKDPSKNDTCKSCWAYWILPIIGAVLLGFLYRYYTSESKSS

SEQ ID NO: 16 - protein, 327 aa, Homo sapiens
MAAAAAAAAATNGTGGSSGMEVDAAVVPSVMACGVTGSVSVALHPLVILNISDHWIRMRSQEGRPVQVIGALIGKQEGRNIEVMNSFELLSHTVEEKIIIDKEYYYTKEEQFKQVFKELEFLGWYTTGGPPDPSDIHVHKQVCEIIESPLFLKLNPMTKHTDLPVSVFESVIDIINGEATMLFAELTYTLATEEAERIGVDHVARMTATGSGENSTVAEHLIAQHSAIKMLHSRVKLILEYVKASEAGEVPFNHEILREAYALCHCLPVLSTDKFKTDFYDQCNDVGLMAYLGTITKTCNTMNQFVNKFNVLYDRQGIGRRMRGLFF

SEQ ID NO: 17 - protein, 343 aa, Homo sapiens
MAQKGVLGPGQLGAVAILLYLGLLRSGTGKEGAAAPCGVAPQARITGGSSAVAGQWPWQVSITYEGVHVCGGSLVSEQWVLSAAHCFPSEHHKEAYEVKLGAHQLDSYSEDAKVSTLKDIIPHPSYLQEGSQGDIALLQLSRPITFSRYIRPICLPAANASFPNGLHCTVTGWGHVAPSVSLLTPKPLQQLEVPLISRETCNCLYNIDAKPEEPHFVQEDMVCAGYVEGGKDACQGDSGGPLSCPVEGLWYLTGIVSWGDACGARNRPGVYTLASSYASWIQSKVTELQPRVVPQTQESQPDSNLCGSHLAFSSAPAQGLLRPILFLPLGLALGLLSPWLSEH

SEQ ID NO: 18 - protein, 198 aa, Homo sapiens
MAPARLFALLLFFVGGVAESIRETEVIDPQDLLEGRYFSGALPDDEDVVGPGQESDDFELSGSGDLDDLEDSMIGPEVVHPLVPLDNHIPERAGSGSQVPTEPKKLEENEVIPKRISPVEESEDVSNKVSMSSTVQGSNIFERTEVLAALIVGGIVGILFAVFLILLLMYRMKKKDEGSYDLGKKPIYKKAPTNEFYA

SEQ ID NO: 19 - protein, 492 aa, Homo sapiens
MEPSSKKLTGRLMLAVGGAVLGSLQFGYNTGVINAPQKVIEEFYNQTWVHRYGESILPTTLTTLWSLSVAIFSVGGMIGSFSVGLFVNRFGRRNSMLMMNLLAFVSAVLMGFSKLGKSFEMLILGRFIIGVYCGLTTGFVPMYVGEVSPTALRGALGTLHQLGIVVGILIAQVFGLDSIMGNKDLWPLLLSIIFIPALLQCIVLPFCPESPRFLLINRNEENRAKSVLKKLRGTADVTHDLQEMKEESRQMMREKKVTILELFRSPAYRQPILIAVVLQLSQQLSGINAVFYYSTSIFEKAGVQQPVYATIGSGIVNTAFTVVSLFVVERAGRRTLHLIGLAGMAGCAILMTIALALLEQLPWMSYLSIVAIFGFVAFFEVGPGPIPWFIVAELFSQGPRPAAIAVAGFSNWTSNFIVGMCFQYVEQLCGPYVFIIFTVLLVLFFIFTYFKVPETKGRTFDEIASGFRQGGASQSDKTPEELFHPLGADSQV

SEQ ID NO: 20 - protein, 180 aa, Homo sapiens
MASTSYDYCRVPMEDGDKRCKLLLGIGILVLLIIVILGVPLIIFTIKANSEACRDGLRAVMECRNVTHLLQQELTEAQKGFQDVEAQAATCNHTVMALMASLDAEKAQGQKKVEELEGEITTLNHKLQDASAEVERLRRENQVLSVRIADKKYYPSSQDSSSAAAPQLLIVLLGLSALLQ

SEQ ID NO: 21 - protein, 341 aa, Homo sapiens
MRKVVLITGASSGIGLALCKRLLAEDDELHLCLACRNMSKAEAVCAALLASHPTAEVTIVQVDVSNLQSVFRASKELKQRFQRLDCIYLNAGIMPNPQLNIKALFFGLFSRKVIHMFSTAEGLLTQGDKITADGLQEVFETNVFGHFILIRELEPLLCHSDNPSQLIWTSSRSARKSNFSLEDFQHSKGKEPYSSSKYATDLLSVALNRNFNQQGLYSNVACPGTALTNLTYGILPPFIWTLLMPAILLLRFFANAFTLTPYNGTEALVWLFHQKPESLNPLIKYLSATTGFGRNYIMTQKMDLDEDTAEKFYQKLLELEKHIRVTIQKTDNQARLSGSCL

SEQ ID NO: 22 - protein, 454 aa, Homo sapiens
MALQLGRLSSGPCWLVARGGCGGPRAWSQCGGGGLRAWSQRSAAGRVCRPPGPAGTEQSRGLGHGSTSRGGPWVGTGLAAALAGLVGLATAAFGHVQRAEMLPKTSGTRATSLGRPEEEEDELAHRCSSFMAPPVTDLGELRRRPGDMKTKMELLILETQAQVCQALAQVDGGANFSVDRWERKEGGGGISCVLQDGCVFEKAGVSISVVHGNLSEEAAKQMRSRGKVLKTKDGKLPFCAMGVSSVIHPKNPHAPTIHFNYRYFEVEEADGNKQWWFGGGCDLTPTYLNQEDAVHFHRTLKEACDQHGPDLYPKFKKWCDDYFFIAHRGERRGIGGIFFDDLDSPSKEEVFRFVQSCARAVVPSYIPLVKKHCDDSFTPQEKLWQQLRRGRYVEFNLLYDRGTKFGLFTPGSRIESILMSLPLTARWEYMHSPSENSKEAEILEVLRHPRDWVR

SEQ ID NO: 23 - protein, 528 aa, Homo sapiens
MRCALALSALLLLLSTPPLLPSSPSPSPSPSQNETATQTTTDSSNKTAPTPASSVTIMATDTAQQSTVPTSKANEILASVKATTLGVSSDSPGTTTLAQQVSGPVNTTVARGGGSGNPTTTIESPKSTKSADTTTVATSTATAKPNTTSSQNGAEDTTNSGGKSSHSVTTDLTSTKAEHLTTPHPTSPLSPRQPTSTHPVATPTSSGHDHLMKISSSSSTVAIPGYTFTSPGMTTTLPSSVISQRTQQTSSQMPASSTAPSSQETVQPTSPATALRTPTLPETMSSSPTAASTTHRYPKTPSPTVAHESNWVTPAGVGTQTRVEEALRQALTHSLLPAGGASDEKLISLICRAVKATFNPAQDKCGIRLASVPGSQTVVVKEITIHTKLPAKDVYERLKDKWDELKEAGVSDMKLGDQGPPEEAEDRFSMPLIITIVCMASFLLLVAALYGCCHQRLSQRKDQQRLTEELQTVENGYHDNPTLEVMETSSEMQEKKVVSLNGELGDSWIVPLDNLTKDDLDEEEDTHL

SEQ ID NO: 24 - protein, 475 aa, Homo sapiens
MAAKSQPNIPKAKSLDGVTNDRTASQGQWGRAWEVDWFSLASVIFLLLFAPFIVYYFIMACDQYSCALTGPVVDIVTGHARLSDIWAKTPPITRKAAQLYTLWVTFQVLLYTSLPDFCHKFLPGYVGGIQEGAVTPAGVVNKYQINGLQAWLLTHLLWFANAHLLSWFSPTIIFDNWIPLLWCANILGYAVSTFAMVKGYFFPTSARDCKFTGNFFYNYMMGIEFNPRIGKWFDFKLFFNGRPGIVAWTLINLSFAAKQRELHSHVTNAMVLVNVLQAIYVIDFFWNETWYLKTIDICHDHFGWYLGWGDCVWLPYLYTLQGLYLVYHPVQLSTPHAVGVLLLGLVGYYIFRVANHQKDLFRRTDGRCLIWGRKPKVIECSYTSADGQRHHSKLLVSGFWGVARHFNYVGDLMGSLAYCLACGGGHLLPYFYIIYMAILLTHRCLRDEHRCASKYGRDWERYTAAVPYRLLPGIF

SEQ ID NO: 25 - protein, 1241 aa, Homo sapiens
MSSAPRRPAKGADSFCTPEPESLGPGTPGFPEQEEDELHRTLGVERFEEILQEAGSRGGEEPGRSYGEEDFEYHRQSSHHIHHPLSTHLPPDARRRKTPQGPGRKPRRRPGASPTGETPTIEEGEEDEDEASEAEGARALTQPSPVSTPSSVQFFLQEDDSADRKAERTSPSSPAPLPHQEATPRASKGAQAGTQVEEAEAEAVAVASGTAGGDDGGASGRPLPKAQPGHRSYNLQERRRIGSMTGAEQALLPRVPTDEIEAQTLATADLDLMKSHRFEDVPGVRRHLVRKNAKGSTQSGREGREPGPTPRARPRAPHKPHEVFVELNELLLDKNQEPQWRETARWIKFEEDVEEETERWGKPHVASLSFRSLLELRRTLAHGAVLLDLDQQTLPGVAHQVVEQMVISDQIKAEDRANVLRALLLKHSHPSDEKDFSFPRNISAGSLGSLLGHHHGQGAESDPHVTEPLMGGVPETRLEVERERELPPPAPPAGITRSKSKHELKLLEKIPENAEATVVLVGCVEFLSRPTMAFVRLREAVELDAVLEVPVPVRFLFLLLGPSSANMDYHEIGRSISTLMSDKQFHEAAYLADEREDLLTAINAFLDCSVVLPPSEVQGEELLRSVAHFQRQMLKKREEQGRLLPTGAGLEPKSAQDKALLQMVEAAGAAEDDPLRRTGRPFGGLIRDVRRRYPHYLSDFRDALDPQCLAAVIFIYFAALSPAITFGGLLGEKTQDLIGVSELIMSTALQGVVFCLLGAQPLLVIGFSGPLLVFEEAFFSFCSSNHLEYLVGRVWIGFWLVFLALLMVALEGSFLVRFVSRFTQEIFAFLISLIFIYETFYKLVKIFQEHPLHGCSASNSSEVDGGENMTWAGARPTLGPGNRSLAGQSGQGKPRGQPNTALLSLVLMAGTFFIAFFLRKFKNSRFFPGRIRRVIGDFGVPIAILIMVLVDYSIEDTYTQKLSVPSGFSVTAPEKRGWVINPLGEKSPFPVWMMVASLLPAILVFILIFMETQITTLIISKKERMLQKGSGFHLDLLLIVAMGGICALFGLPWLAAATVRSVTHANALTVMSKAVAPGDKPKIQEVKEQRVTGLLVALLVGLSIVIGDLLRQIPLAVLFGIFLYMGVTSLNGIQFYERLHLLLMPPKHHPDVTYVKKVRTLRMHLFTALQLLCLALLWAVMSTAASLAFPFILILTVPLRMVVLTRIFTDREMKCLDANEAEPVFDEREGVDEYNEMPMPV

SEQ ID NO: 26 - protein, 707 aa, Homo sapiens
MAAAVAAAGRLGWLFAALCLGNAAGEAAPGPRVLGFCLEEDGAAGAGWVRGGAARDTPDATFLLRLFGPGFANSSWSWVAPEGAGCREEAASPAGEWRALLRLRLRAEAVRPHSALLAVRVEPGGGAAEEAAPPWALGLGAAGLLALAALARGLQLSALALAPAEVQVLRESGSEAERAAARRLEPARRWAGCALGALLLLASLAQAALAVLLYRAAGQRAVPAVLGSAGLVFLVGEVVPAAVSGRWTLALAPRALGLSRLAVLLTLPVALPVGQLLELAARPGRLRERVLELARGGGDPYSDLSKGVLRCRTVEDVLTPLEDCFMLDASTVLDFGVLASIMQSGHTRIPVYEEERSNIVDMLYLKDLAFVDPEDCTPLSTITRFYNHPLHFVFNDTKLDAVLEEFKRGKSHLAIVQKVNNEGEGDPFYEVLGLVTLEDVIEEIIRSEILDESEDYRDTVVKRKPASLMAPLKRKEEFSLFKVSDDEYKVTISPQLLLATQRFLSREVDVFSPLRISEKVLLHLLKHPSVNQEVRFDESNRLATHHYLYQRSQPVDYFILILQGRVEVEIGKEGLKFENGAFTYYGVSALTVPSSVHQSPVSSLQPIRHDLQPDPGDGTHSSAYCPDYTVRALSDLQLIKVTRLQYLNALLATRAQNLPQSPENTDLQVIPGSQTRLLGEKTTTAAGSSHSRPGVPVEGSPGRNPGV

SEQ ID NO: 27 - protein, 501 aa, Homo sapiens
MVRKPVVSTISKGGYLQGNVNGRLPSLGNKEPPGQEKVQLKRKVTLLRGVSIIIGTIIGAGIFISPKGVLQNTGSVGMSLTIWTVCGVLSLFGALSYAELGTTIKKSGGHYTYILEVFGPLPAFVRVWVELLIIRPAATAVISLAFGRYILEPFFIQCEIPELAIKLITAVGITVVMVLNSMSVSWSARIQIFLTFCKLTAILIIIVPGVMQLIKGQTQNFKDAFSGRDSSITRLPLAFYYGMYAYAGWFYLNFVTEEVENPEKTIPLAICISMAIVTIGYVLTNVAYFTTINAEELLLSNAVAVTFSERLLGNFSLAVPIFVALSCFGSMNGGVFAVSRLFYVASREGHLPEILSMIHVRKHTPLPAVIVLHPLTMIMLFSGDLDSLLNFLSFARWLFIGLAVAGLIYLRYKCPDMHRPFKVPLFIPALFSFTCLFMVALSLYSDPFSTGIGFVITLTGVPAYYLFIIWDKKPRWFRIMSEKITRTLQIILEVVPEEDKL

SEQ ID NO: 28 - protein, 905 aa, Homo sapiens [sequence omitted]

SEQ ID NO: 29 - protein, 474 aa, Homo sapiens
MELPSGPGPERLFDSHRLPGDCFLLLVLLLYAPVGFCLLVLRLFLGIHVFLVSCALPDSVLRRFVVRTMCAVLGLVARQEDSGLRDHSVRVLISNHVTPFDHNIVNLLTTCSTVSESEAESATGRFPGAQLKAPLSPLAFPMEDTELPLTPILYPTCQFFFIFLNIFLLAFSSPGSQPLLNSPPSFVCWSRGFMEMNGRGELVESLKRFCASTRLPPTPLLLFPEEEATNGREGLLRFSSWPFSIQDVVQPLTLQVQRPLVSVTVSDASWVSELLWSLFVPFTVYQVRWLRPVHRQLGEANEEFALRVQQLVAKELGQTGTRLTPADKAEHMKRQRHPRLRPQSAQSSFPPSPGPSPDVQLATLAQRVKEVLPHVPLGVIQRDLAKTGCVDLTITNLLEGAVAFMPEDITKGTQSLPTASASKFPSSGPVTPQPTALTFAKSSWARQESLQERKQALYEYARRRFTERRAQEAD

SEQ ID NO: 30 - protein, 532 aa, Homo sapiens
MEKSNETNGYLDSAQAGPAAGPGAPGTAAGRARRCAGFLRRQALVLLTVSGVLAGAGLGAALRGLSLSRTQVTYLAFPGEMLLRMLRMIILPLVVCSLVSGAASLDASCLGRLGGIAVAYFGLTTLSASALAVALAFIIKPGSGAQTLQSSDLGLEDSGPPPVPKETVDSFLDLARNLFPSNLVVAAFRTYATDYKVVTQNSSSGNVTHEKIPIGTEIEGMNILGLVLFALVLGVALKKLGSEGEDLIRFFNSLNEATMVLVSWIMWYVPVGIMFLVGSKIVEMKDIIVLVTSLGKYIFASILGHVIHGGIVLPLIYFVFTRKNPFRFLLGLLAPFATAFATCSSSATLPSMMKCIEENNGVDKRISRFILPIGATVNMDGAAIFQCVAAVFIAQLNNVELNAGQIFTILVTATASSVGAAGVPAGGVLTIAIILEAIGLPTHDLPLILAVDWIVDRTTTVVNVEGDALGAGILHHLNQKATKKGEQELAEVKVEAIPNCKSEEETSPLVTHQNPAGPVASAPELESKESVL

SEQ ID NO: 31 - protein, 910 aa, Homo sapiens
MKKMSRNVLLQMEEEEDDDDGDIVLENLGQTIVPDLGSLESQHDFRTPEFEEFNGKPDSLFFNDGQRRIDFVLVYEDESRKETNKKGTNEKQRRKRQAYESNLICHGLQLEATRSVLDDKLVFVKVHAPWEVLCTYAEIMHIKLPLKPNDLKNRSSAFGTLNWFTKVLSVDESIIKPEQEFFTAPFEKNRMNDFYIVDRDAFFNPATRSRIVYFILSRVKYQVINNVSKFGINRLVNSGIYKAAFPLHDCKFRRQSEDPSCPNERYLLYREWAHPRSIYKKQPLDLIRKYYGEKIGIYFAWLGYYTQMLLLAAVVGVACFLYGYLNQDNCTWSKEVCHPDIGGKIIMCPQCDRLCPFWKLNITCESSKKLCIFDSFGTLVFAVFMGVWVTLFLEFWKRRQAELEYEWDTVELQQEEQARPEYEARCTHVVINEITQEEERIPFTAWGKCIRITLCASAVFFWILLIIASVIGIIVYRLSVFIVFSAKLPKNINGTDPIQKYLTPQTATSITASIISFIIIMILNTIYEKVAIMITNFELPRTQTDYENSLTMKMFLFQFVNYYSSCFYIAFFKGKFVGYPGDPVYWLGKYRNEECDPGGCLLELTTQLTIIMGGKAIWNNIQEVLLPWIMNLIGRFHRVSGSEKITPRWEQDYHLQPMGKLGLFYEYLEMIIQFGFVTLFVASFPLAPLLALVNNILEIRVDAWKLTTQFRRLVPEKAQDIGAWQPIMQGIAILAVVTNAMIIAFTSDMIPRLVYYWSFSVPPYGDHTSYTMEGYINNTLSIFKVADFKNKSKGNPYSDLGNHTTCRYRDFRYPPGHPQEYKHNIYYWHVIAAKLAFIIVMEHVIYSVKFFISYAIPDVSKRTKSKIQREKYLTQKLLHENHLKDMTKNMGVIAERMIEAVDNNLRPKSE

SEQ ID NO: 32 - protein, 879 aa, Homo sapiens
MGRLASRPLLLALLSLALCRGRVVRVPTATLVRVVGTELVIPCNVSDYDGPSEQNFDWSFSSLGSSFVELASTWEVGFPAQLYQERLQRGEILLRRTANDAVELHIKNVQPSDQGHYKCSTPSTDATVQGNYEDTVQVKVLADSLHVGPSARPPPSLSLREGEPFELRCTAASASPLHTHLALLWEVHRGPARRSVLALTHEGRFHPGLGYEQRYHSGDVRLDTVGSDAYRLSVSRALSADQGSYRCIVSEWIAEQGNWQEIQEKAVEVATVVIQPSVLRAAVPKNVSVAEGKELDLTCNITTDRADDVRPEVTWSFSRMPDSTLPGSRVLARLDRDSLVHSSPHVALSHVDARSYHLLVRDVSKENSGYYYCHVSLWAPGHNRSWHKVAEAVSSPAGVGVTWLEPDYQVYLNASKVPGFADDPTELACRVVDTKSGEANVRFTVSWYYRMNRRSDNVVTSELLAVMDGDWTLKYGERSKQRAQDGDFIFSKEHTDTFNFRIQRTTEEDRGNYYCVVSAWTKQRNNSWVKSKDVFSKPVNIFWALEDSVLVVKARQPKPFFAAGNTFEMTCKVSSKNIKSPRYSVLIMAEKPVGDLSSPNETKYIISLDQDSVVKLENWTDASRVDGVVLEKVQEDEFRYRMYQTQVSDAGLYRCMVTAWSPVRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD